(19) United States
(12) Patent Application Publication
(10) Pub. No.: US 2026/0273074 A1
(43) Pub. Date: Sep. 17, 2026

(54) FERRITIN-TARGETING PROTACS AND METHODS OF INDUCING PYROPTOSIS

(71) Applicant: Wisconsin Alumni Research Foundation, Madison, WI (US)

(72) Inventors: Quanyin Hu, Madison, WI (US); Yu Chen, Madison, WI (US)

(73) Assignee: Wisconsin Alumni Research Foundation, Madison, WI (US)

(21) Appl. No.: 19/470,940

(22) PCT Filed: Mar. 28, 2024
(86) PCT No.: PCT/US2024/021881

§ 371 Date: Sep. 30, 2025

Related U.S. Application Data
(60) Provisional application No. 63/493,893, filed on Apr. 3, 2023.

(51) Int. Cl. A61K 47/55; A61K 47/54; A61K 47/69; A61P 35/00

(52) U.S. Cl. CPC A61K 47/55; A61K 47/542; A61K 47/545; A61K 47/6931; A61P 35/00

(57) ABSTRACT

As described herein, a ferritin-targeting PROTAC has the structure Ferritin Binder-linker-von Hippel-Lindau (VHL) ligand. Also described are nanoparticles including the PROTAC and albumin, for example, pharmaceutical compositions comprising the nanoparticles, and methods of a primary or metastatic tumor, a cancer tissue, or a liquid cancer with elevated ferritin expression compared to normal tissues in a subject in need thereof by administering the compositions.

### CROSS-REFERENCE TO RELATED APPLICATIONS

This application claims priority to U.S. Provisional Application 63/493,893 filed on Apr. 3, 2023, which is incorporated herein by reference in its entirety.

### FIELD OF THE DISCLOSURE

The present disclosure is related to a novel proteolysis targeting chimera (PROTAC) for inducing rapid iron accumulation in cells and its application in nanomedicine for pyroptosis-mediated anticancer treatment.

### BACKGROUND

Malignant cells frequently reprogram an overactivated but sophisticated system to control iron content, forming the dynamic equilibrium of elemental iron among influx, efflux, usage, and storage to support cell growth and proliferation. Recent studies provide abundant insights into the roles of iron metabolism in malignancies, and thus various ferric therapeutics are developed for cancer treatment. However, little research has been designed to investigate the response of cancer cells to intracellular iron stress in terms of the sharp iron excess or deficiency.

Although exploring the behaviors of cancer cells against iron imbalance is beneficial for expanding our understanding of ferric homeostasis and correspondingly developing anticancer therapeutics, currently limited chemical and biological techniques pose challenges to establishing an acute status with iron overload or exhaustion inside malignant cells in a short period. Commonly used approaches to reduce or increase the intracellular available iron content rely on chelators or exogenous supplements, which could be classified as the direct strategy. Iron chelators with poor membrane permeability possess limited capacity to induce an iron plummet. Since cellular uptake of iron ions is under strict regulation, incubating cancer cells with an iron-containing medium usually achieves a slow elevation in iron content. Moreover, the disruption of the related signaling pathways by employing chemical or biological tools, serving as an indirect method to induce intracellular iron imbalance, is often flawed in the efficiency and practicability of triggering iron stress. Taking ferritin that mineralizes and stores the intracellular iron ions as an example, genetically or chemically intervening in this protein expression or functionality is expected to cause the overload of iron ions. However, apart from the druggability uncertainty of ferritin, small-molecule inhibitors that need to durably bind to the active pockets of functional proteins usually cause partial inhibitory effects with higher concentrations. Even though gene editing technologies hold the potential to selectively deplete ferritin by blocking its transcription and translation, the time-consuming operation procedures and acting processes impose additional obstacles to these genetic strategies for creating an acute condition of ferritin destruction.

What is needed are novel anticancer therapeutics and their use to induce rapid iron accumulation in cells resulting in programmed cell death of cancer cells.

### BRIEF SUMMARY

In an aspect, a ferritin-targeting PROTAC has the structure Ferritin Binder-linker-von Hippel-Lindau (VHL) ligand.

In another aspect, included is a nanoparticle composition, comprising the above-described PROTACs, as well as pharmaceutical compositions comprising the nanoparticles. An exemplary nanoparticle for the nanoparticle composition is an albumin nanoparticle.

In an aspect, a method of treating a primary or metastatic tumor, a cancer tissue, or a liquid cancer with elevated ferritin expression compared to normal tissues in a subject in need thereof comprises administering the compositions described herein.

### BRIEF DESCRIPTION OF THE DRAWINGS

FIGS. 1A-D illustrate the discovery of ferritin-targeting PROTAC DeFer-2. (1A) Chemical structures of oleic acid (OA)-based von Hippel-Lindau (VHL)-recruiting PROTACs. (1B) Inverse isothermal titration calorimetry (ITC) profile of ferritin titrated into DeFer-2 at 30° C. to determine the binding kinetics of the degrader. (1C) Immunoblots for ferritin and β-actin of B16F10 cells treated with DeFer-2 at 0.5 μM for 0, 4, 8, 12, 16, and 24 h. (1D) Immunoblots for ferritin and β-actin of B16F10 cells treated with DeFer-2 at 0, 0.008, 0.04, 0.2, 1, and 5 μM for 12 h.

FIG. 2 shows the synthetic route of DeFer-1, 2, 3, and DeFer-2epi. (i) N-(tert-butoxycarbonyl)glycine, 4-(tert-butoxycarbonylamino)butyric acid, or N-tert-butyloxycarbonyl-6-amino-hexanoic acid, HATU, TEA, DMF, rt; (ii) oleoyl chloride, TEA, DMF, 0° C.→rt.

FIG. 3 shows ITC profile of ferritin titrated into oleic acid at 30° C. to determine the binding kinetics of oleic acid.

FIGS. 4A and B show immunoblots. (4A) immunoblots for ferritin and β-actin of B16F10 cells treated with DeFer-1 or DerFer-3 at 0.5 μM for 0, 4, 8, 12, 16, and 24 h. (4B) Immunoblots for ferritin and β-actin of B16F10 cells treated with DeFer-1 or DerFer-3 at 0, 0.008, 0.04, 0.2, 1, and 5 μM for 12 h.

FIGS. 5A-F show the mechanism of action of DeFer-2-mediated ferritin degradation. (5A) Immunoblots for ferritin and β-actin of B16F10 cells treated with DeFer-2 (0.5 μM), DeFer-2epi (0.5 μM), the combination of VHL ligand (0.5 μM) and OA (0.5 μM), and DeFer-2 (0.5 μM) combined with pretreatment (1 h) of proteasome inhibitor Epox (1 μM) for 12 h. (5B) Immunoblots for ferritin immunoprecipitated by the anti-VHL antibody in the lysate of B16F10 cells treated with DeFer-2 (5 μM) or DeFer-2epi (5 μM) for 2 h. (5C) Superposition of the feasible ferritin dimer-DeFer-2-VHL ternary complexes after the conformational alignment of the aminobutyryl linker. (5D) Overlap of four relatively stable ferritin dimer-DeFer-2-VHL states during a 200-ns molecular dynamics simulation of the selected ternary complex. Proteins are displayed as cartoon representations, and DeFer-2 is shown as spheres. (5E) Overall surface representation of ferritin dimer-DeFer-2-VHL ternary complex in the fourth state generated from the above 200-ns molecular dynamics simulation and a close-up of interactions between DeFer-2 and the proteins. (5F) Volcano plots of the −log₁₀ (P value) versus the log 2-fold change presenting the abundance changes of global proteins in B16F10 cells after DeFer-2 (0.5 μM) treatment versus DeFer-2epi (0.5 μM) for 12 h. The plot indicates the ferritin and the proteins with substantial abundance reduction compared to ferritin are labeled.

FIG. 6 shows immunoblots for ferritin and β-actin of B16F10 cells treated with DeFer-2 (0.5 μM) or a combination of DeFer-2 (0.5 μM) and VHL ligand (50 μM) for 12 h.

FIGS. 7A-C show docking of ferritin complexes. (7A) Landscape of symmetric docking score and ligand conformer energy for the feasible ternary complexes. The pose with the lowest score and energy was selected, as indicated by the dotted circle. The dotted circle indicates the selected optimal ternary mode. (7B) Landscape of symmetric pair RMSD values for the four relatively stable ferritin dimer-DeFer-2-VHL states. (7C) Overlap of ferritin dimer-DeFer-2-VHL ternary complex with the VH032-bound VHL (PDB: 4W9H) and arachidonic acid-bound ferritin dimer (PDB: 4DE6).

FIGS. 8A-H show DeFer-2 rapidly elevates intracellular free iron content to trigger pyroptosis in cancer cells. (8A) Intracellular free iron content in B16F10 cells after DeFer-2 (5 μM) treatment for 8 h. Data are presented as mean±SD (n=3), *P<0.05, analyzed by unpaired Student's t-test. (8B) The ROS level in B16F10 cells after the treatment with DeFer-2 (5 μM), DeFer-2epi (5 μM), or the combination of VHL ligand (5 μM) and OA (5 μM) for 8 h. Scale bar, 50 μm. (8C) Relative viabilities of B16F10 cells treated with DeFer-2, DeFer-2epi, or the combination of VHL ligand and OA at various concentrations for 24 or 48 h. Data are presented as mean±SD (n=4). (8D) Morphology changes of B16F10 cells after the treatment with DeFer-2 (5 μM), DeFer-2epi (5 μM), or the combination of VHL ligand (5 μM) and OA (5 μM) for 24 h. Arrows indicate pyroptotic cells. Scale bar, 50 μm. (8E) LDH release from B16F10 cells after the indicated treatments for 24 h. Data are presented as mean±SD (n=3). (8F) Relative viabilities of B16F10 cells pretreated with DeFer-2 (5 μM) for 4 h followed by the combination treatment of DFOM (25 μM) and DeFer-2 (5 μM) for the remaining 20 h. Data are presented as mean±SD (n=4). (8G) Relative viabilities of B16F10 cells pretreated with Z-VAD (10 μM), NSCI (0.1 μM), or VX-765 (10 μM) for 4 h followed by the combination treatments of DeFer-2 (5 μM) and Z-VAD (10 μM), NSCI (0.1 μM), or VX-765 (10 μM) for another 24 h. Data are presented as mean±SD (n=5). (8H) Immunoblots for cleaved caspase 3 or GSDME-N in B16F10 cells after the indicated DeFer-2 treatments with or without GSH pretreatment for 4 h. For caspase 3 detection, cell lysates are collected at 8 h after the treatments, and for GSDME-N, cell lysates are collected at 20 h after the treatments. *P<0.05, **P<0.01, ***P<0.001, n.s. indicated no significance, analyzed by One-Way ANOVA followed by Tukey's multiple comparisons test.

FIG. 9 shows LDH release from B16F10 cells pretreated with NSCI (0.1 μM) or VX-765 (10 μM) for 4 h followed by the combination treatments of DeFer-2 (5 μM) and NSCI (0.1 μM) or VX-765 (10 μM) for another 24 h. Data are presented as mean±SD (n=3), n.s. indicated no significance, ***P<0.001, analyzed by One-Way ANOVA followed by Tukey's multiple comparisons test.

FIGS. 10A-E show aDeFer-2 induces cytotoxicity in vitro and inhibits tumor growth in vivo. (10A) Size distribution of aDeFer-2 in water and saline. The inset image showed the morphology of aDeFer-2 observed with TEM. Scale bar, 100 nm. (10B) Immunoblots for ferritin and β-actin of B16F10 cells treated with aDeFer-2 at 0, 0.008, 0.04, 0.2, 1, and 5 μM for 12 h. (10C) Immunoblots for ferritin and β-actin of B16F10 cells treated with aDeFer-2 (0.5 μM), aDeFer-2epi (0.5 μM), and DeFer-2 (0.5 μM) combined with pretreatment (1 h) of Epox (1 μM) for 12 h. (10D) Morphology changes of B16F10 cells after aDeFer-2 (5 μM) or aDeFer-2epi (5 μM) treatment for 24 h. Arrows indicate pyroptotic cells. Scale bar, 50 μm. (10E) B16F10 tumor growth curves after indicated treatments. (10F) Survival curves of B16F10 tumor-bearing mice after indicated treatments. (10G) Body weight changes of B16F10 tumor-bearing mice during the therapeutic period. For (10E), (10F), and (10G), data are presented as mean±standard error of mean (SEM) (n=6), n.s. indicated no significance, **P<0.01, ***P<0.001.

FIGS. 11A-D shows particle size distributions. (11A) Size distribution of aDeFer-2 before (suspended in water) and after lyophilization (resuspended in saline). (11B) Size and PDI changes of aDeFer-2 incubated in ultrapure water at 4° C. for 10 days (n=3). (11C) Accumulative DeFer-2 release profiles from aDeFer-2 after incubation with PBS containing 10% FBS at 37° C. Data are presented as mean±SD (n=3). (11D) Size distribution of aDeFer-2epi in water or saline. The inset image shows the morphology of aDeFer-2epi observed with TEM. Scale bar, 100 nm.

FIG. 12 shows relative viabilities of B16F10 cells treated with aDeFer-2 or aDeFer-2epi at various concentrations for 24 or 48 h. Data are presented as mean±SD (n=4).

FIGS. 13A and B show immunoblots. (13A) Immunoblots for cleaved caspase 3 and β-actin of B16F10 cells treated with aDeFer-2 for 8 h at the indicated concentrations with or without GSH pre-treatment for 4 h. (13B) Immunoblots for cleaved N-terminal GSDME (GSDME-N) and β-actin of B16F10 cells treated with aDeFer-2 for 20 h at the indicated concentrations with or without GSH pre-treatment for 4 h.

FIGS. 14A and B show H&E staining and hematological analysis. (14A) H&E staining of major organs (heart, liver, spleen, lung, and kidney) to evaluate the safety of aDeFer-2 treatments on tumor-bearing mice. Scale bar, 100 μm. (14B) Hematological analysis of tumor-bearing mice following intravenous administration of saline or aDeFer-2. Data are presented as mean±SD (n=3), n.s. indicated no significance. WBC, white blood cell; LYM, Lymphocytes; MON, Monocytes; NEU, Neutrophils; RBC, red blood cell; HGB, hemoglobin; HCT, hematocrit; MCV, mean corpuscular volume; MCH, mean corpuscular hemoglobin; MCHC, mean corpuscular hemoglobin concentration; PLT, platelets.

FIG. 15 is a schematic illustration of anticancer therapies based on the ferritin degrader-based pyroptosis inducer.

The above-described and other features will be appreciated and understood by those skilled in the art from the following detailed description, drawings, and appended claims.

### DETAILED DESCRIPTION

Proteolysis targeting chimeras (PROTACs) have been developed to recognize and degrade proteins of interest (POIs) by redirecting the ubiquitin-proteasome system (UPS). Given the unique mechanism of action, this modular platform employs heterobifunctional chimeras composed of functional ligands for POIs and E3 ligases that are bridged by a short linker to rapidly induce posttranslational knock-down of various target proteins, leading to loss of function without noticeable effect on genome and transcriptome. Therefore, coupled with the features of high knock-down efficiency and easy operation, these small molecules are feasible to serve as excellent tools for creating a stress status for investigations on cellular functions and intracellular biological processes at a molecular level.

In this study, the PROTAC platform was used to develop a ferritin degrader for creating iron overload stress, based on the hypothesis that rapid degradation of ferritin would efficiently induce the intracellular accumulation of accessible iron ions. Oleic acid (OA) was employed as the binder for a ferritin dimer benefiting from the involvement of unsaturated long-chain fatty acids in the assembling process among these identical iron-storage proteins. The widely used ligand for E3 ligase, VH032, was selected to construct the von Hippel-Lindau (VHL)-recruiting PROTACs. Since the linkers that bridge the two binders can affect the degradation potency of PROTACs, three alkyl chains with different lengths were incorporated into the VH032-OA pair. According to the degradation efficacy, the screened construct DeFer-2 was selected to elevate intracellular free iron content. As shown herein, iron excess stress induced by DeFer-2 could trigger caspase 3-gasdermin E (GSDME)-mediated pyroptosis in cancer cells. To maximize its therapeutic potential, a tailored albumin nano-formulation was prepared to encapsulate DeFer-2, realizing favorable anticancer treatment efficacy in B16F10 subcutaneous tumor-bearing mice without noticeable side effects.

In an aspect, a ferritin-targeting PROTAC has the structure Ferritin Binder-linker-von Hippel-Lindau (VHL) ligand.

Exemplary Ferritin Binders include a C₁₄ to C₂₄ unsaturated fatty acid, or a haloether anesthetic. C₁₄ to C₂₄ unsaturated fatty acids include oleate and arachidonate. Haloether anesthetics include halothane, isoflurane, and propofol.

In an aspect, the linker is an alkyl linker having the formula —NH(CH₂)ₙC(═O)—, wherein n is 1 to 20. In an aspect, n is 1 to 3 in the —NH(CH₂)ₙC(═O)— linker.

In another aspect, the linker is a PEG linker of the formula —(O—CH₂—CH₂)ₘ— or —(O—CH₂—CH₂)ₘ—O—, wherein m has a value from 2 to 20.

Combined alkyl/PEG linkers may also be employed.

In an aspect, the VHL ligand is one of the following:

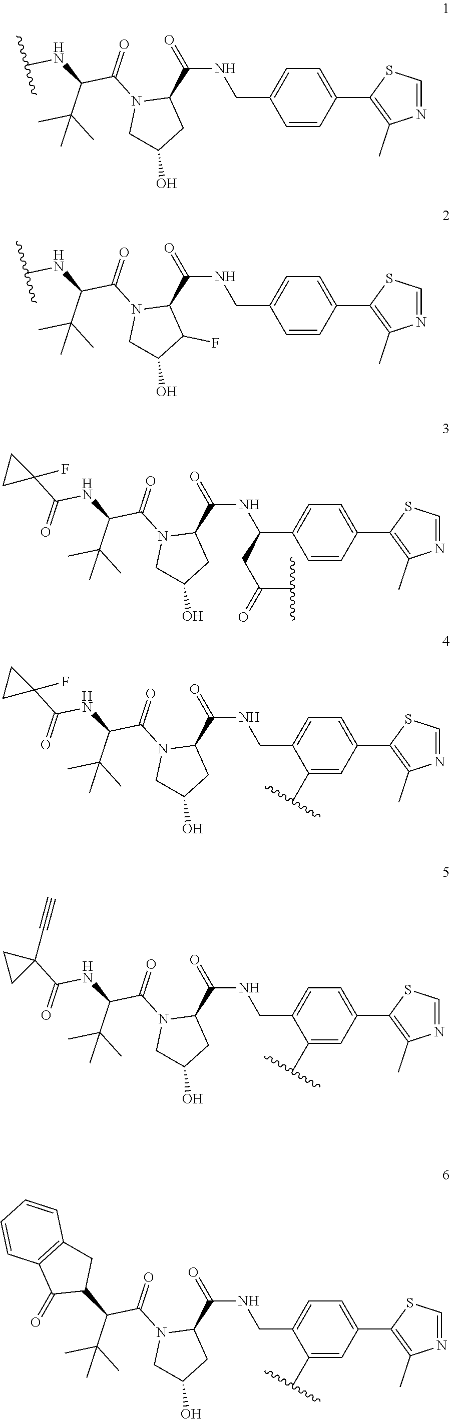 [SMILES: *N[C@@H](C(=O)N1C[C@@H](O)C[C@@H]1C(=O)NCc1ccc(-c2scnc2C)cc1)C(C)(C)C ; *N[C@@H](C(=O)N1C[C@@H](O)C(F)[C@@H]1C(=O)NCc1ccc(-c2scnc2C)cc1)C(C)(C)C ; *C(=O)C[C@@H](NC(=O)[C@H]1C[C@H](O)CN1C(=O)[C@H](NC(=O)C1(F)CC1)C(C)(C)C)c1ccc(-c2scnc2C)cc1 ; *c1cc(-c2scnc2C)ccc1CNC(=O)[C@H]1C[C@H](O)CN1C(=O)[C@H](NC(=O)C1(F)CC1)C(C)(C)C ; *c1cc(-c2scnc2C)ccc1CNC(=O)[C@H]1C[C@H](O)CN1C(=O)[C@H](NC(=O)C1(C#C)CC1)C(C)(C)C ; *c1cc(-c2scnc2C)ccc1CNC(=O)[C@H]1C[C@H](O)CN1C(=O)[C@H](C1Cc2ccccc2C1=O)C(C)(C)C]

In an aspect, a nanoparticle composition comprises a nanoparticle and the PROTAC as described above.

Exemplary nanoparticles include organic nanoparticles, inorganic nanoparticles, liposome nanoparticles, virus-like nanoparticles, and polymeric nanoparticles. Organic nanoparticles include albumin nanoparticles. Inorganic nanoparticles include, without limitation, nanoparticles comprising iron or silica. Liposome nanoparticles include, without limitation, conventional liposomes, sterically-stabilized liposomes, ligand-targeted liposomes, and combinations thereof. Polymeric nanoparticles include, without limitation, nanoparticles comprising chitosan, PLGA, PEI, and combinations thereof.

In an aspect, the nanoparticle comprises albumin and the PROTAC as described above. Exemplary nanoparticles have diameters of 10 to 100 nm, specifically, 5 to 500 nm and more specifically 125 to 200 nm. In an aspect, the nanoparticles have a polydispersity of 0.08 to 0.25.

In an aspect, the albumin is human serum albumin. In some embodiments, the molar ratio of the albumin to the PROTAC is 1:7 to 1:1, specifically 1:2 to 1:1. After formation, the nanoparticles may be dried, such as lyophilized, and resuspended in a carrier.

In an aspect, the nanoparticles are suspended, dissolved, or emulsified in a carrier such as water. In some embodiments, the composition is sterile filterable. In some embodiments, the composition is a buffered solution. Exemplary carrier solutions include sterile water, saline, phosphate-buffered saline, 5% dextrose in water solution, Ringer's solution, and Ringer's lactate solution.

In an aspect, the PROTAC can be loaded in platelets, platelet-derived microparticles, or tumor-derived extracellular vesicles for tumor-targeted delivery.

In an aspect, the PROTAC may be loaded into or covalently linked to a platelet cell or a platelet membrane via a chemical linker moiety, specifically a bifunctional linker.

In an embodiment, the platelet cell is a human platelet cell. In one embodiment, the platelet cell is an autologous platelet cell. Platelet cells can be isolated from whole blood by centrifuging whole blood and separating the platelet-rich plasma. The platelets can then be separated from the plasma by centrifugation.

The PROTAC may be conjugated to the platelet cells. In order to conjugate the PROTAC to the platelets, the PROTAC can be chemically modified with, for example, a bifunctional linker. In an embodiment, the bifunctional linker includes SMCC (succinimidyl-4-(N-maleimidomethyl)cyclohexane-1-carboxylate), MBS (m-maleimidobenzoyl-N-hydroxysuccinimide ester), sulfo-MBS (m-maleimidobenzoyl-N-hydroxysulfosuccinimide ester), GMBS (N-γ-maleimidobutyryloxysuccinimide ester), sulfo-GMBS (N-γ-Maleimidobutyryloxysulfosuccinimide ester, EMCH (N-(ε-maleimidocaproic acid) hydrazide), EMCS (N-(ε-maleimidocaproyloxy) succinimide ester), sulfo-EMCS N-(ε-maleimidocaproyloxy) sulfo succinimide ester), PMPI (N-(ρ-maleimidophenyl) isocyanate), SIAB (N-succinimidyl(4-iodoacetyl)aminobenzoate), SMPB (succinimidyl 4-(ρ-maleimidophenyl) butyrate), sulfo-SIAB (N-sulfosuccinimidyl(4-iodoacetyl)aminobenzoate), sulfo-SMCC (sulfosuccinimidyl 4-(N-maleimidomethyl)cyclohexane-1-carboxylate), sulfo-SMBP (sulfo succinimidyl 4-(ρ-maleimidophenyl) butyrate), EDC (1-ethyl-3-(3-dimethylaminopropyl) carbodiimide hydrochloride), or MAL-PEGSCM (maleimide PEG succinimidyl carboxymethyl). In an embodiment, the bifunctional linker comprises SMCC or an NHS ester. SMCC is a hetero-bifunctional linker that contains N-hydroxysuccinimide (NHS) ester and maleimide groups that allow covalent conjugation of amine- and sulfhydryl-containing molecules. NHS esters react with primary amines at pH 7-9 to form amide bonds, while maleimides react with sulfhydryl groups at pH 6.5-7.5 to form stable thioether bonds.

In an aspect, the PROTAC is in the form of a platelet-derived microparticle. Platelet-derived microparticles (PMPs) are small particles generated from the membranes of intact platelets also known as platelet-derived microvesicles. PMPs can be formed as a result of platelet activation from interactions between platelets and plastic storage containers during platelet processing and storage. Platelets can also be activated by thrombin, collagen, ADP, Ca²⁺, complement components, shear stress, and the like.

Tumor-derived extracellular vesicles for tumor-targeted delivery. Extracellular vesicles (EVs) are kinds of two-layer vesicles secreted by cells. Tumor-derived EVs (TDEVs) are related to the tumor microenvironment and cancer development and can be used as a natural drug carrier with high tumor targeting and permeability. Techniques such as ultracentrifugation, size exclusion chromatography, ultrafiltration, flow field-flow fractionation, polymer precipitation, immunoaffinity chromatography, and microfluidics methods can be used to isolate TDEVs. Loading of the PROTACs into the TDEVs can include incubation, electroporation, sonication, extrusion, and freeze-thaw methods, as well as attachment of PROTACSs to the surface of the TEDEVs using chemical conjugation methods or binding to surface-expressed ligands.

In an aspect, the composition is suitable for injection. In some aspects, the composition is suitable for intravenous administration. In some embodiments, the composition is administered intraperitoneally, intraarterially, intrapulmonarily, orally, by inhalation, intravesicularly, intramuscularly, intratracheally, subcutaneously, intraocularly, intrathecally, intratumorally, or transdermally.

Pyroptosis, a type of programmed cell death mediated by gasdermin proteins, is characterized by the continuous expansion of cells forming large ballooning bubbles until the cell membrane ruptures, resulting in the release of cellular contents and subsequent activation of a strong inflammatory response. As an important innate immune response in the body, pyroptosis plays a crucial role in antagonizing infection and endogenous danger signals. Moreover, the latest research reveals that cytotoxic lymphocytes rely on gasdermin-mediated pyroptosis to kill tumor cells, suggesting that pyroptosis is also closely involved in anti-cancer immune response and rising as a very promising method for cancer treatment.

Thus, a method of treating a primary or metastatic tumor, cancer tissue or liquid cancer comprises administering the nanoparticle composition comprising the PROTAC as described herein.

The compositions and methods described herein are particularly useful to treat tumors, tissues and liquid cancers with elevated ferritin expression compared to normal tissues, such as colorectal cancer tumors, lung cancer tumors, breast cancer tumors, melanoma tissues, prostate tumors, liposarcomas, gastric tumors, ovarian tumors, thyroid tumors, liver tumors, colon tumors, glioblastomas, esophageal tumors, leukemias, endometrial tumors, gliomas, adenocarcinomas, neuroblastomas, cervical tumors, and the like.

The invention is further illustrated by the following non-limiting examples.

### EXAMPLES

### Methods

Reagents, antibodies, cells, and mice: (2S,4R)-1-((S)-2-amino-3,3-dimethylbutanoyl)-4-hydroxy-N-(4-(4-methylthiazol-5-yl)benzyl) pyrrolidine-2-carboxamide hydrochloride (VHL ligand), (2S,4S)-1-((S)-2-Amino-3,3-dimethylbutanoyl)-4-hydroxy-N-(4-(4-methylthiazol-5-yl)benzyl) pyrrolidine-2-carboxamide hydrochloride 4-(tert-butoxycarbonylamino)butyric acid and N-(tert-butoxycarbonyl)glycine were purchased from AmBeed (Arlington Hts, IL, USA). Oleoyl chloride was provided by Tokyo Chemical Industry (TCI). O-(7-Azabenzotriazol-1-yl)-N,N,Nzzhlxy,Nzzhlxy-tetramethyluronium hexafluorophosphate (HATU), N-tert-butyloxycarbonyl-6-amino-hexanoic acid was bought from Chem-Impex (Wood Dale, IL, USA). Oleic acid and Liproxstatin-1 analog were provided by Cayman Chemical (Ann Arbor, MI, USA). VX-765 and Z-VAD(OMe)-FMK (Z-VAD) were provided by TargetMol (Wellesley Hills, MA, USA). Epoxomicin was bought from MedChemExpress (Monmouth Junction, NJ, USA). Apoferritin from equine spleen (A3660), NSCI, 2,7-dichlorodihydrofluorescein diacetate (DCFH-DA), triethylamine, and all the other chemical reagents and solvents were obtained from Sigma-Aldrich (St. Louis, MO, USA). Cell Counting Kit-8 (CCK-8) kit were acquired from APExBIO (Houston, TX, USA). CytoScan™ LDH Cytotoxicity Assay kit was obtained from G-Biosciences (St. Louis, MO, USA). Pierce™ Protein A/G Magnetic Beads (88802) were bought from Thermo Fisher Scientific (Waltham, MA USA). The primary antibody against cleaved caspase 3 (9661T) was provided by Cell Signaling Technology (Danvers, MA, USA), and the mouse monoclonal IgG₁ κ anti-VHL antibody (sc-135657) was purchased from Santa Cruz Biotechnology (Dallas, TX, USA). Other primary antibodies, including anti-cleaved N-terminal DFNA5/GSDME (ab222407), anti-ferritin (ab75973), anti-β-actin (ab49900) antibodies and secondary antibodies used in western blotting assay were obtained from Abcam (Cambridge, UK). All antibody dilutions were performed according to the manufacturer's guidance.

The murine B16F10 cell line was purchased from ATCC. Cells were cultured in the CO₂ incubator (Fisher) at 37° C. with 5% CO₂ and 90% relative humidity. The 12-week-old C57BL/6 male mice were purchased from the Jackson Laboratory. The animal study protocol was approved by the Institutional Animal Care and Use Committee (IACUC) at the University of Wisconsin-Madison.

Western blot analysis: After various treatments, ferritin expression levels in B16F10 cells were evaluated to investigate the DeFer-2-mediated ferritin degradation. Briefly, B16F10 cells were seeded in 6-well culture plates (5×10⁴ cells per well) and cultured for 24 h. Then the medium in the corresponding wells except the negative control was replaced with 1 mL of medium containing ferritin degraders, albumin nanoparticles, the epimer formulations, the combination of VHL ligand and oleic acid, or the combination of the degrader and VHL ligand at the indicated concentrations. After incubation for 12 h, cells were washed with PBS and lysed with RIPA lysis buffer containing phenylmethylsulfonyl fluoride (PMSF, 1 mM) and the phosphatase inhibitor cocktail for 30 min. The lysates were centrifuged at 12,000 rpm for 10 min at 4° C., followed by quantification of the total proteins in the supernatant through the bicinchoninic acid assay (BCA) assay. Afterward, the proteins in the supernatant were mixed with the loading buffer and heated at 95° C. for 15 min. Equal amounts of proteins from different samples were loaded into 15% SDS-polyacrylamide gel (20 μg protein per lane) to separate target proteins. After separation, proteins were transferred to polyvinylidene fluoride (PVDF) membranes before the membranes were blocked with 5% nonfat milk in PBST. Then proteins were incubated with primary antibodies overnight at 4° C., followed by the incubation with the goat-anti-rabbit IgG H&L (HRP) secondary antibodies at room temperature for 2 h. Finally, the bands were detected with electrochemiluminescence (ECL) western blot substrate for imaging, and ImageJ software was used for quantification.

As for the determination of cleaved-GSDME and cleaved-caspase-3, B16F10 cells were seeded in 6-well culture plates (2×10⁵ cells per well) and cultured for 24 h. Then the medium in the corresponding wells except the negative control was replaced with 1 mL of medium containing ferritin degraders, albumin nanoparticles, or the degrader with inhibitors at the indicated concentrations. Incubation for another 6 or 8 h was used to test the cleaved-caspase-3, while treatment for another 16 or 20 h was used to test cleaved-GSDME. The following procedures were the same as mentioned above.

Isothermal Titration calorimetry: The binding kinetics profiles of oleic acid and DeFer-2 to ferritin were measured by V P-Differential Scanning calorimeter (MicroCal). As for the ferritin proteins, the apoferritin from equine spleen (A3660, Sigma-Aldrich) that is not combined with iron was used. Before the ITC test, 1 mL of apoferritin in glycerol solution (50% glycerol and 0.075 M NaCl) was placed in a dialysis bag with a molecular weight cut-off of 3 kDa. The dialysis bags were then immersed in the prepared solution (20 mM Tris-HCl, pH 8.5, and 130 mM NaCl) and stirred at room temperature. The dialysis buffer was changed every 24 h for three times, and the last buffer was collected and 0.05% DMSO was added to serve as the ITC buffer. As for the oleic acid, 12 μM apoferritin in the ITC buffer was placed in the ITC cell, and the syringe was full of 250 μM oleic acid in the same ITC buffer. Besides the first injection with 5 μL of the oleic acid solution, 18 aliquots with 10 μL of the oleic acid solution were titrated into the apoferritin solution with a time interval of 300 s. Since the relatively low solubility of DeFer-2 in the ITC buffer, inverse ITC titration was adopted by injecting a 10 μL of the aliquot of apoferritin (100 μM) in the ITC buffer into the cell containing 10 μM DeFer-2 in the same ITC buffer. Besides the first injection with 1 μL of the apoferritin solution, 25 aliquots were titrated with a time interval of 300 s. Raw data were corrected for control titrations (the solution in the syringe into the blank ITC buffer in the cell) and integrated using Origin 7.0 (MicroCal). Binding kinetics parameters, including N, Kd, ΔH, and ΔS, were calculated using a single-class binding site model.

Quantitative proteomics analysis: Cell culture and proteomic sample preparation: B16F10 cells were seeded in T75 cell culture flasks (2×10⁶ cells per well) and cultured for 24 h. Then the medium in the corresponding flasks was replaced with 15 mL of medium containing DeFer-2 (0.5 μM) or DeFer-2epi (0.5 μM). After incubation for 12 h, cells were washed with cold PBS twice and digested with 2 mL trypsin. The cell pellets were collected in 1.5 mL Eppendorf tubes through centrifugation at 1200 rpm for 3 minutes at 4° C. As for the proteomic sample preparation, cell pellets placed on the ice were completely resuspended with a cold 100 mM ammonium bicarbonate (ABC) solution. Then, a 23-gauge needle attached to a 1 mL syringe was used to homogenize each cell pellet sample by drawing the cells into the syringe and then fully depressing the plunger. After being homogenized six times, the lysates were centrifugated at 15,000 g for 20 min at 4° C. The supernatants were transferred to new tubes, and a small aliquot of each lysate supernatant was collected to perform the BCA assay. Based on the quantification results, an appropriate volume of lysate supernatant containing 20 μg proteins was collected and incubated with 10 mM DL-dithiothreitol (DTT) at 37° C. for 30 min. Then, the iodoacetamide (IAA) stock solution was directly added to each sample for a final solution with 55 mM IAA. The protein solutions were incubated at 37° C. for another 45 min in the dark. Then trypsin (0.1 g/L in 250 mM ABC solution) was added at a ratio of 1:20 (enzyme/target protein) for 18-h digestion, which was halted by adding 10% formic acid. The peptides for each sample were dried to zero volume in the speed vacuum concentrator and cleaned up on a ZipTip® C18 tip (Millipore) according to the manufacturer's protocol.

LC-MS/MS analysis: The LC separations were performed with a Waters™ Nano-Acquity UPLC System. All samples were resuspended in 0.1% formic acid in water at around 0.1 μg/μL, and 0.2 μL total peptide mass solution was injected twice on column with a 140 min increasing gradient for each sample. The mobile phases (A: water with 0.1% formic acid and B: acetonitrile with 0.1% formic acid) were driven, and gradient elution was performed at 0.350 μL/min. Mobile phase B was increased from the initial 3% to 5% in 1 min, followed by an increase to 30% at 110 min, a ramp to 95% B at 115 min, and a wash at 95% B for 10 min. Flow was then ramped back to 3% B in 3 min, and the column was re-equilibrated at 3% B for 10 min, for a total 140-min run. Eluted peptides were analyzed on Thermo Orbitrap Q-Exactive™ MS operated in positive ion mode with nano-ESI high-resolution Q-Exactive™ analysis. The full scan was performed in the range of 300-2000 m/z at a resolution of 70,000, and the AGC target was set to 10⁶ with a maximal injection time of 100 ms. Following the full MS scan, a microscan was performed with a resolution of 17,500, a dynamic exclusion of 7 s, an AGC target of 10⁵, a maximal injection time of 100 ms, a loop count of 10, an isolation window of 3.5 m/z, NCE of 30. A 50 min blank was run between each sample to check for carry-over.

Data analysis: All the resulting raw files were analyzed using Proteome Discoverer software v.2.4 (Thermo Fisher Scientific), SEQUEST HT using Uniprot Mus musculus reference proteome with a decoy database added to establish control variability and false discovery rates. The ANOVA model is used for statistical significance analysis to determine the significant differences between different treatments.

Co-Immunoprecipitation assay: B16F10 cells were seeded in T75 cell culture flasks (2×10⁶ cells per well) and cultured for 24 h. Then the medium in the corresponding flasks, except the control, was replaced with 15 mL of medium containing DeFer-2 (5 μM) or DeFer-2epi (5 μM). After incubation for 2 h, cells were washed with cold PBS and lysed on ice with IP lysis buffer (50 mM Tris-HCl, pH 7.4; 150 mM NaCl; 2 mM EDTA; 1% NP-40) containing phenylmethylsulfonyl fluoride (PMSF, 1 mM) and the phosphatase inhibitor cocktail for 30 min. The lysates were collected and centrifuged at 10,000 g for 10 minutes at 4° C. On the one hand, 10 μL of lysate supernatants were saved as the input samples. On the other hand, 25 μL of magnetic beads was added to each supernatant in microcentrifuge tubes, and the resulting mixtures were incubated at 4° C. with gentle rotation for 30 min. Next, the sample-containing tube was placed on a magnetic rack for 1 minute, and the supernatant was transferred into a new microcentrifuge tube placed on ice, serving as the precleared samples. The total proteins in the precleared samples were quantified through the BCA assay. Each precleared lysate was added an appropriate amount of mouse monoclonal IgG₁ κ anti-VHL antibody (sc-135657, Santa Cruz Biotechnology) and then incubated at 4° C. for 2 hours with gentle rotation. After that, the pre-washed magnetic bead (equal to 25 μL of magnetic beads) was added to each sample. The resulting mixtures were incubated at room temperature for 1 h with gentle rotation. Subsequently, the supernatant was discarded by placing the tubes on a magnetic rack, and the obtained magnetic beads were washed with IP lysis buffer for 3 times. Finally, an appropriate amount of WB loading buffer was added to each magnetic bead-containing tube and input sample. The boiled samples were subjected to WB examination for ferritin or VHL proteins.

Procedures for in silico modeling of DeFer-2-mediated ternary complex: Protein-protein docking was performed following the previously reported method with the Rosetta software suite. The open-source cheminformatics software, RDKit (version 2020.03.1), was used to generate a conformer. Python was used to build PROTAC ternary models.

Starting structure: The structures of VHL and ferritin (ferritin dimer-arachidonic acid: 4DE6 and VHL-VH032: 4W9H) were downloaded from the RCSB PDB. The orientation direction (data not shown) was adjusted manually with PyMol. The starting structure for docking was prepared with Rosetta after generating the ligand params.

Protein-protein docking: The docking_protocol.mpi_linuxgccrelease program was used to generate 10,000 diverse binding modes. The “-partners ABX_YC” flag indicated that the small-molecule ligands and paired proteins must move together. The search space was defined by “-dock_pert” flag. Afterward, the decoy with the distance between two linker atoms smaller than 8 Å (data not shown) was selected for further evaluation through the InterfaceAnalyzer module in Rosetta.

Linker conformation: RDKit was used to generate 10,000 conformations (threshold >0.5) were generated with RDKit. After that, depending on the energy (low to high), 216 conformers were selected (data not shown).

Ternary models: A custom python script was applied to evaluate the RMSD of the stubs in given linker conformer relative to their location in a docked model. A specified cutoff value was applied to evaluate the RMSD of the stubs. If it passed the cutoff value, then a complete model of the ternary complex will be built. The best-performed model was selected for the following molecular dynamics simulation.

Molecular dynamics (MD) simulation: Protein-protein docking and molecular dynamics simulation were applied to screen the most stable ternary structure. A TIP3P water model was established to build the water box, and chlorine or sodium ions were incorporated for system neutralization. The molecular dynamics simulation was run on an AMBER package with leaprc.protein.ff14SB as the force field for the protein, and General Amber Force field (GAFF) as the force field for DeFer-2.

An energy minimization was first performed with the purpose of generating a starting conformation with a low energy. The steepest descent method (4000 steps) was first performed, followed by a conjugation gradient method (4000 steps). Afterward, protein, ligand, water, and ions were minimized. Then, only protein and ligand were minimized. The whole system was then heated under canonical ensemble (0-310 K, 300 ps, with Langevin thermostat), and the force constant for the harmonic restraint was set to 10.0 kcal mol Å. After the equilibration of the system (10 ns, constant pressure: 1.0 bar, relaxation time for barostat bath: 2.0 ps), the final simulation of the product was run for 100 ns under NPT (periodic boundary condition, time step: 2 fs). A SHAKE algorithm was used to constrain the bonds connected with hydrogen atoms. The particle-mesh Ewald (PME) method was applied to handle the long-range electrostatics. An 8.0 Å was set as the cut-off value for short range interactions.

The pairwise root mean square deviation of atoms was calculated with the “ptraj” tool in AMBER 20, aiming to analyze complexes stability and the trajectories of the sampling method that was clustered into 4 groups by a k-means method installed “cpptraj” tool. A similar conformation of the structures in the same groups was determined by that average distance between each group is around 2 Å. The represent structure in the main group, containing most structures, was selected as the most stable mode for the detailed analysis.

Determination of free iron content: Here, the focus was on detecting the intracellular free iron ions rather than protein-bound iron contents. The procedures for sample preparation were based on a previously reported method. B16F10 cells were seeded in T75 cell culture flasks (2×10⁶ cells per well) and cultured for 24 h. Then the medium in the corresponding flasks, except the control, was replaced with 15 mL of medium containing 5 μM of DeFer-2. After incubation for 8 h, cells were harvested and resuspended in 1 mL PBS. Besides, 800 μL of PBS cell suspension was subjected to centrifugation, and 780 μL of the supernatant was carefully removed. Afterward, 280 μL of ultrapure water was added to the cell precipitate. Following shaking at horizontal rotators for 12 h, the supernatant was transferred into 30 KDa ultrafiltration centrifuge tube after twice centrifugation at 2,000 g for 20 min. The sample in the ultrafiltration tube underwent centrifugation at 3,500 g for 20 min, and the filtrate was collected for the iron content detection. The remaining 200 μL PBS cell suspension was subjected to centrifugation, and 190 μL supernatant was carefully removed. Afterward, the cell pellet was treated with 50 μL RIPA lysis buffer containing phenylmethylsulfonyl fluoride (PMSF, 1 mM) and the phosphatase inhibitor cocktail for 30 min. The lysates were centrifuged at 12,000 rpm for 10 min at 4° C., followed by quantification of the total proteins in the supernatant through the BCA assay.

As for iron content determination, 200 μL of the abovementioned filtrates were collected and treated with 10 μL of hydrochloric acid solution (6 mol/L), 100 μL of acetic acid-sodium acetate buffer (pH=4.5), 10 μL of 10% aqueous hydroxylamine hydrochloride, and 100 μL of o-phenanthroline (1 mg/mL). After shaking at horizontal rotators for 30 min, the samples were transferred to a 96-well plate. The absorbance was measured by a microplate reader (Infinite M Plex) at the detection wavelength of 510 nm. The iron ion content could be calculated according to the standard curve. Finally, the intracellular free iron content was presented as iron weight per mg protein.

ROS detection: DCFH-DA was used to evaluate the generation of intracellular ROS in cancer cells. 5×10⁴ B16F10 cells were seeded in confocal dishes and cultured for 24 h at 37° C. Then the medium in the corresponding wells, except the control, was replaced with 1 mL of medium containing DeFer-2 (5 μM), DeFer-2epi (5 μM), or the combination of VHL ligand (5 μM) and oleic acid (5 μM). After incubation for 8 h, cells were stained with DCFH-DA (10 μM) for 30 min. Finally, the cells were washed with PBS and subjected to Leica SP8 Confocal WLL STED Microscope for observation.

Cell viability evaluation: The cell viability of B16F10 cells treated with ferritin degraders with or without various inhibitors was evaluated by CCK-8 assay. Briefly, 5×10³ cells were seeded in 96-well plates and cultured for 24 h for attachment. Then the medium in the corresponding wells, except the control, was replaced with 100 μL of medium containing the indicated degrader formulations with concentrations of 0.008, 0.04, 0.2, 1, and 5 μM. After incubation for 24 or 48 h, CCK-8 reagent was added and interacted with cells at 37° C. for 1 h. The absorbance was measured by a microplate reader (Infinite M Plex) at the detection wavelength of 450 nm.

For blocking assays with Z-VAD, NSCI, and VX-765, 5×10³ cells were seeded in 96-well plates and cultured 24 h for attachment. Then the medium in the corresponding wells, except the control, was replaced with 100 μL of medium containing Z-VAD (10 μM), NSCI (0.1 μM), or VX-765 (10 μM). After incubation for 4 h, the medium in the corresponding wells, except the inhibitor control, was replaced with 100 μL of medium containing Z-VAD (10 μM), NSCI (0.1 μM), or VX-765 (10 μM) with DeFer-2 (5 μM), respectively. Following another 24-h incubation, CCK-8 reagents were added and interacted with cells at 37° C. for 1 h. The absorbance was measured by a microplate reader (Infinite® M Plex) at the detection wavelength of 450 nm. For blocking assays with DFOM, 5×10³ cells were seeded in 96-well plates and cultured 24 h for attachment. Then the medium in the corresponding wells, except the control, was replaced with 100 μL of medium containing DeFer-2 (5 μM). After incubation for 4 h, the medium in the corresponding wells was replaced with 100 μL of medium containing DeFer-2 (5 μM) and DFOM (25 μM). Following another 20-h incubation, CCK-8 reagents were added and interacted with cells at 37° C. for 1 h. The absorbance was measured by a microplate reader (Infinite® M Plex) at the detection wavelength of 450 nm.

Morphology observation: 5×10⁴ B16F10 cells were seeded in confocal dishes and cultured for 24 h at 37° C. Then the medium in the corresponding wells, except the control, was replaced with 1 mL of medium containing DeFer-2 (5 μM), DeFer-2epi (5 μM), the corresponding albumin formulations, or the combination of VHL ligand (5 μM) and oleic acid (5 μM). After incubation for 24 h, the cells were washed with PBS and subjected to a Nikon Eclipse Ti Intensilight fluorescence microscope in a phase contrast mode for observation.

LDH release assay: 5×10³ cells were seeded in 96-well plates and cultured 24 h for attachment. Then the medium in the corresponding wells, except the control, was replaced with 100 μL of medium containing the indicated degrader formulations with concentrations of 1, 5, and 10 μM. After incubation for 24 h, the cell samples were used to measure LDH release according to the manufacturer's instructions of CytoScan™ LDH Cytotoxicity Assay kit from G-Biosciences (St. Louis, MO, USA).

For blocking assays with NSCI and VX-765, 5×10³ cells were seeded in 96-well plates and cultured 24 h for attachment. Then the medium in the corresponding wells, except the control, was replaced with 100 μL of medium containing NSCI (0.1 μM) or VX-765 (10 μM). After incubation for 4 h, the medium in the corresponding wells, except the inhibitor control, was replaced with 100 μL of medium containing NSCI (0.1 μM) or VX-765 (10 μM) with DeFer-2 (5 μM), respectively. Following another 24-h incubation, the cell samples were used to measure LDH release according to the manufacturer's instructions of CytoScan™ LDH Cytotoxicity Assay kit from G-Biosciences (St. Louis, MO, USA).

Preparation and characterization of aDeFer-2 and DeFer-2epi: A solution of DeFer-2 or DeFer-2epi in ethanol (1000 μL at 0.58 mg/mL) was added dropwise into albumin bovine V aqueous solution (2000 μL at 12.5 mg/mL). The mixture was stirred for 30 minutes at room temperature and then transferred into a 10 kDa ultrafiltration centrifuge tube. After centrifugation at 4,500 rpm for 20 min, 5 mL of saline was added to the solution in the tube, which was subjected to centrifugation at 4,500 rpm for 20 min. The addition of 5 mL of saline and the following centrifugation was repeated three times. The obtained mixture in the tube was the albumin-DeFer-2 or albumin-DeFer-2epi formulation resuspended in saline.

Z-average diameter and PDI of aDeFer-2 or aDeFer-2epi formulation were measured by DLS (Zetasizer, Nano-ZS, Malvern, UK). The morphology of aDeFer-2 or aDeFer-2epi was characterized using TEM (FEI Tecnai T-12 Cryo TEM). aDeFer-2 or aDeFer-2epi (1 mg/mL) was added to the carbon-coated grid. After water evaporation, the grid was subjected to TEM observation.

To analyze encapsulation efficiency (EE) and loading efficiency (LC), the formulation was dissolved in acetonitrile (0.1 mol/L) and then subjected to the high performance liquid chromatography (HPLC) system as detected at 254 nm. All samples were injected with a volume of 10 μL. The mobile phases (A: water with 0.05% ammonium acetate and B: acetonitrile) were driven with a constant ratio (A/B=63.6/36.4) at 1.4 mL/min for a total 20-min run.

The encapsulation efficiency (EE) and loading capacity (LC) were determined according to Equation (1) and (2) (n=3):

$$EE ( \% ) = \frac{amount of DeFer - 2 or DeFer - 2 epi in the formulation}{total amount of DeFer - 2 or DeFer - 2 epi added} \qquad ( 1 )$$

$$LC ( \% ) = \frac{amount of DeFer - 2 or DeFer - 2 epi in the formulation}{formulation weight} \qquad ( 2 )$$

As for evaluating the stability of aDeFer-2, aDeFer-2 in water was prepared as the abovementioned method by changing saline with ultrapure water to perform the centrifugation three times. The obtained mixture was subjected to lyophilization (SP VirTis Freezemobile™) for 48 h and resuspended in saline. Before and after lyophilization, the sizes of aDeFer-2 in water and aDeFer-2 in saline were determined by DLS, respectively. Another aDeFer-2 in water generated by the same method was placed at 4° C. and subjected to size examination every two days.

Regarding DeFer-2 release from aDeFer-2, we added aDeFer-2 to PBS containing 10% FBS to test DeFer-2 release from the albumin nanoparticles in the plasma. In detail, 50 μL of aDeFer-2 in saline containing about 70 μg DeFer-2 (the real content of DeFer-2 in every sample determined by HPLC) was placed into 450 μL PBS containing 10% FBS and then incubated under agitation (100 rpm) at 37° C. At pre-designed time intervals, including 0.5, 1, 2, 4, 8, 12, and 24 h, tubes were centrifuged at 14,800 rpm for 40 min at 4° C. The amount of released DeFer-2 was calculated by determining the DeFer-2 contents in the supernatant. 100 μL of the supernatant from each sample was added to tubes containing 400 μL acetonitrile, and then tubes were centrifuged at 14,800 rpm for 20 min at 4° C. after rough vortex. The final supernatant was collected and subjected to the abovementioned HPLC analysis.

Evaluation of tumor growth inhibition, survival, and biosafety: B16F10 cells were collected and resuspended in a PBS buffer solution containing Matrigel (50%). Male C₅₇BL/6J mice (8 weeks old) were anesthetized and injected with 50 μL cell suspensions (1×10⁶ cells) at the right flank. On day 7 after implantation, when the tumor volumes reached approximately 100 mm³, tumor-bearing C₅₇BL/6J mice were randomly assigned into 3 groups (n=6) and then i.v. injected with albumin vehicle saline, aDeFer-2 (10 mg/kg), aDeFer-2epi (10 mg/kg), and aDeFer-2 (10 mg/kg) plus intratumoral injection of NSCI (0.4 mg/kg) every other day four times. The tumor volumes and body weights were monitored daily during the treatment period. As for survival, we set the tumor volume over 2000 mm³ as the endpoint for tumor-bearing mice.

To further verify the biosafety profile of aDeFer-2, the same tumor-bearing mice (n=3) were established and i.v. injected saline or aDeFer-2 as the same treatment schedules. At 24 h of the last administration, the major organs were harvested for H&E staining. Furthermore, the blood samples were collected for hematological analysis using AbaxisHM5 Complete Blood Count (CBC) Analyzer.

Statistical analysis: Statistical analysis was performed using GraphPad Prism (version 9). All in vitro data are presented as mean±standard deviation (SD). All in vivo data are presented as mean±standard error of mean (SEM). Unpaired student's t-test was used for between two-group comparison and ANOVA was used to perform multiple-group analysis. Survival studies were analyzed using Log-rank test. Differences were considered statistically significant if P<0.05; *P<0.05, **P<0.01, ***P<0.001.

### Example 1: Chemical Synthesis and Characterization

Synthesis and characterization of ferritin degraders (DeFer-1, 2, 3 and DeFer-2epi): Step 1:(2S,4R)-1-((S)-2-amino-3,3-dimethylbutanoyl)-4-hydroxy-N-(4-(4-methylthiazol-5-yl)benzyl) pyrrolidine-2-carboxamide hydrochloride or (2S,4S)-1-((S)-2-Amino-3,3-dimethylbutanoyl)-4-hydroxy-N-(4-(4-methylthiazol-5-yl)benzyl) pyrrolidine-2-carboxamide hydrochloride (100 mg, 0.214 mmol) and 1.2 mole equivalent (0.256 mmol) of N-(tert-butoxycarbonyl)glycine (for the intermediate of DeFer-1), 4-(tert-butoxycarbonylamino)butyric acid (for the intermediate of DeFer-2), or N-tert-butyloxycarbonyl-6-amino-hexanoic acid (for the intermediate of DeFer-3) were dispersed in anhydrous N,N-dimethylformamide (DMF, 2 mL) in a flask. Then, After the addition of triethylamine (120 μL, 0.856 mmol), HATU (204 mg, 0.536 mmol) was added in one portion. Following stirring 30 minutes at room temperature, the reaction mixture was diluted with 30 mL of distilled water. The aqueous phase was extracted with ethyl acetate (EA). Afterward, the organic phase was collected, washed with brine, and dried over anhydrous sodium sulfate. The filtrate was collected by filtration and concentrated under the reduced pressure. Finally, the residue was purified by column chromatography on silica gel (dichloromethane/EA/methanol=6/6/1, v/v/v) to yield colorless oily products, the intermediates of DeFer-1 (yield: 85.9%), 2 (yield: 78.9%), 3 (yield: 82.7%), and 2epi (yield: 76.8%). The chemical structures were confirmed by ¹H-NMR (400 MHz) in CDCl₃ and ESI mass spectrum.

DeFer-1 intermediate: ¹H NMR (400 MHz, Chloroform-d) δ 8.67 (s, 1H), 7.59 (s, 1H), 7.35 (s, 4H), 7.00 (s, 1H), 5.50 (s, 1H), 4.76 (s, 1H), 4.50 (d, J=8.9 Hz, 2H), 4.35 (dd, J=15.1, 5.4 Hz, 1H), 4.12 (q, J=7.1 Hz, 1H), 4.04 (d, J=11.6 Hz, 1H), 3.77 (s, 1H), 3.71 (dd, J=16.8, 6.4 Hz, 1H), 3.68-3.62 (m, 1H), 2.51 (s, 3H), 2.43 (s, 1H), 2.25-2.14 (m, 1H), 1.43 (s, 9H), 0.95 (s, 9H). ¹³C NMR (101 MHz, Chloroform-d) δ 171.17, 170.14, 168.95, 157.87, 150.27, 148.46, 138.16, 131.54, 130.83, 129.46, 127.96, 117.31, 80.63, 70.54, 60.41, 58.65, 57.58, 44.26, 43.15, 37.68, 36.59, 35.44, 28.27, 26.36, 21.06, 16.05, 14.20. HRMS (ESI, m/z): [M+H]⁺ calcd for C₂₉H₄₂N₅O₆S⁺ 588.285032. found 588.28477.

DeFer-2 intermediate: ¹H NMR (400 MHz, Chloroform-d) δ 8.68 (s, 1H), 7.47 (s, 1H), 7.40-7.31 (m, 4H), 7.10 (d, J=7.9 Hz, 1H), 4.77 (dd, J=16.4, 8.3 Hz, 2H), 4.57 (dd, J=14.9, 6.6 Hz, 1H), 4.49 (s, 1H), 4.43 (d, J=7.9 Hz, 1H), 4.33 (dd, J=15.0, 5.1 Hz, 1H), 4.12 (q, J=7.1 Hz, 2H), 3.58 (d, J=11.9 Hz, 1H), 3.23-3.05 (m, 2H), 2.51 (s, 3H), 2.22 (d, J=8.6 Hz, 2H), 2.15 (t, J=11.1 Hz, 1H), 1.75 (d, J=7.9 Hz, 2H), 1.42 (s, 9H), 0.96 (s, 9H). ¹³C NMR (101 MHz, Chloroform-d) δ 173.59, 171.17, 170.74, 156.70, 150.28, 148.49, 138.18, 131.62, 130.95, 129.52, 128.15, 79.80, 70.17, 60.40, 58.24, 56.74, 43.24, 39.32, 35.84, 34.54, 33.07, 28.41, 26.84, 26.46, 21.06, 16.05, 14.20. HRMS (ESI, m/z): [M+H]⁺ calcd for C₃₁H₄₆N₅O₆S⁺ 616.316332. found 616.31693.

DeFer-2epi intermediate: ¹H NMR (400 MHz, Chloroform-d) δ 8.69 (s, 1H), 7.58 (s, 1H), 7.42-7.31 (m, 5H), 6.73 (d, J=8.3 Hz, 1H), 5.54 (d, J=9.8 Hz, 1H), 4.74 (d, J=9.0 Hz, 2H), 4.64 (dd, J=14.9, 7.0 Hz, 1H), 4.47 (td, J=8.6, 7.9, 3.5 Hz, 2H), 4.30 (dd, J=14.9, 5.1 Hz, 1H), 4.12 (q, J=7.1 Hz, 1H), 3.97 (d, J=8.4 Hz, 1H), 3.81 (d, J=10.9 Hz, 1H), 3.15 (d, J=6.3 Hz, 2H), 2.52 (s, 3H), 2.36 (d, J=14.2 Hz, 1H), 2.27-2.15 (m, 3H), 1.77 (td, J=6.8, 4.2 Hz, 2H), 1.43 (s, 9H), 1.26 (t, J=7.1 Hz, 2H), 0.94 (s, 9H). ¹³C NMR (101 MHz, Chloroform-d) δ 172.77, 172.66, 172.37, 156.49, 150.37, 148.56, 137.37, 131.49, 131.25, 129.63, 128.19, 71.16, 60.40, 59.87, 58.63, 57.58, 43.50, 39.58, 35.03, 34.73, 33.32, 28.40, 26.56, 26.37, 21.05, 16.06, 14.20. HRMS (ESI, m/z): [M+H]⁺ calcd for C₃₁H₄₆N₅O₆S⁺ 616.316332. found 616.31325.

DeFer-3 intermediate: ¹H NMR (400 MHz, Chloroform-d) δ 8.68 (s, 1H), 7.36 (d, J=2.3 Hz, 5H), 6.18 (d, J=8.7 Hz, 1H), 4.73 (q, J=10.6, 9.3 Hz, 2H), 4.61-4.48 (m, 3H), 4.35 (dd, J=15.0, 5.3 Hz, 1H), 4.12 (q, J=7.1 Hz, 2H), 3.60 (d, J=11.4 Hz, 1H), 3.07 (d, J=6.7 Hz, 2H), 2.52 (s, 3H), 2.19 (s, 4H), 1.94 (m, 1H), 1.67-1.54 (m, 2H), 1.41 (s, 9H), 0.94 (s, 9H). ¹³C NMR (101 MHz, Chloroform-d) δ 173.76, 171.17, 170.71, 158.70, 150.29, 149.39, 138.38, 131.60, 130.95, 129.52, 128.13, 115.87, 79.50, 70.08, 60.40, 58.45, 57.52, 43.24, 40.50, 36.24, 35.86, 34.78, 29.65, 28.43, 26.43, 25.17, 21.06, 16.05, 14.20. HRMS (ESI, m/z): [M+H]⁺ calcd for C₂₉H₅₀N₅O₆S⁺ 644.347632. found 644.34787.

Step 2: In a round-bottom flask, the above intermediates (0.15 mmol) were dissolved in dichloromethane (DCM, 2 mL). Trifluoroacetic acid (TFA, 2 mL) was added and the mixture was stirred at room temperature for 6 h, leading to the removal of the tert-butyloxycarbonyl group. After DCM and TFA were removed by rotary evaporation, toluene (2 mL) was added to the residuals and was removed by rotary evaporation. The treatment with toluene was repeated 3 times. The resulting oily materials and TEA (0.45 mmol) were dispersed in anhydrous DMF (3 mL) at 0° C. Then, oleoyl chloride (0.18 mmol) dissolved in 1 mL of anhydrous DMF was added dropwise. After stirring overnight at room temperature, the reaction mixture was diluted with 20 mL of distilled water. The aqueous phase was extracted with EA, and the combined organic phase was washed with brine and dried over anhydrous sodium sulfate. The filtrate was collected by filtration and concentrated under reduced pressure. The residue was purified by column chromatography on silica gel (DCM/methanol=15/1, v/v) to yield colorless oily products, DeFer-1 (yield: 38.1%), 2 (yield: 29.1%), 3 (yield: 46.2%), and 2epi (yield: 33.4%). The chemical structures were confirmed by ¹H-NMR (400 MHz) in CDCl₃ and ESI mass spectrum.

DeFer-1: ¹H NMR (400 MHz, Chloroform-d) δ 8.67 (s, 1H), 7.91 (t, J=5.9 Hz, 1H), 7.40 (d, J=9.1 Hz, 1H), 7.33 (q, J=8.3 Hz, 4H), 6.59 (t, J=4.5 Hz, 1H), 5.42-5.27 (m, 2H), 4.78 (t, J=8.2 Hz, 1H), 4.67 (d, J=9.2 Hz, 1H), 4.60-4.49 (m, 2H), 4.30 (dd, J=15.1, 5.3 Hz, 1H), 4.03-3.85 (m, 3H), 3.71 (dd, J=11.2, 3.6 Hz, 1H), 2.51 (s, 3H), 2.35 (ddd, J=13.0, 8.4, 4.4 Hz, 1H), 2.24 (dd, J=13.7, 8.1 Hz, 1H), 2.17 (dd, J=8.7, 6.6 Hz, 2H), 2.00 (q, J=6.6 Hz, 4H), 1.64-1.52 (m, 2H), 1.37-1.21 (m, 21H), 0.98 (s, 9H), 0.91-0.84 (m, 3H). ¹³C NMR (101 MHz, Chloroform-d) δ 174.24, 171.20, 171.18, 169.13, 150.30, 148.46, 138.26, 131.54, 130.89, 130.06, 129.67, 129.46, 127.91, 70.24, 58.65, 57.59, 57.42, 43.09, 37.28, 36.36, 35.73, 31.91, 29.77, 29.73, 29.53, 29.34, 29.32, 29.29, 29.16, 27.24, 27.19, 26.51, 25.68, 22.69, 16.08, 14.13. HRMS (ESI, m/z): [M+Na]⁺ calcd for C₄₂H₆₅N₅O₅SNa⁺ 774.459862. found 774.46129.

DeFer-2: ¹H NMR (400 MHz, Chloroform-d) δ 8.68 (s, 1H), 7.44-7.31 (m, 5H), 6.96 (d, J=8.2 Hz, 1H), 5.85 (t, J=6.1 Hz, 1H), 5.42-5.28 (m, 2H), 4.76 (t, J=8.1 Hz, 1H), 4.56 (dd, J=14.9, 6.6 Hz, 1H), 4.50 (s, 1H), 4.47 (d, J=8.2 Hz, 1H), 4.33 (dd, J=15.0, 5.3 Hz, 1H), 4.13 (d, J=11.4 Hz, 1H), 3.58 (dd, J=11.4, 3.5 Hz, 1H), 3.32 (dq, J=13.4, 6.6 Hz, 1H), 3.26-3.16 (m, 1H), 2.59-2.53 (m, 1H), 2.52 (s, 3H), 2.26-2.19 (m, 2H), 2.19-2.11 (m, 3H), 2.00 (d, J=6.3 Hz, 4H), 1.83-1.69 (m, J=7.1 Hz, 2H), 1.59 (t, J=7.4 Hz, 2H), 1.36-1.21 (m, 21H), 0.95 (s, 9H), 0.91-0.84 (m, 3H). ¹³C NMR (101 MHz, Chloroform-d) δ 174.00, 173.38, 172.16, 170.72, 150.32, 148.46, 138.14, 131.61, 130.97, 130.04, 129.71, 129.54, 128.14, 70.15, 58.26, 56.80, 43.25, 38.31, 36.80, 35.94, 34.74, 33.11, 31.91, 29.77, 29.73, 29.53, 29.33, 29.32, 29.29, 29.17, 27.24, 27.18, 26.47, 26.11, 25.79, 22.69, 16.04, 14.13. HRMS (ESI, m/z): [M+H]⁺ calcd for C₄₄H₇₀N₅O₅S⁺ 780.509218. found 780.51264.

DeFer-2epi: ¹H NMR (400 MHz, Chloroform-d) δ 8.68 (s, 1H), 7.54 (t, J=6.0 Hz, 1H), 7.41-7.32 (m, 5H), 6.70 (d, J=8.6 Hz, 1H), 5.84 (t, J=5.8 Hz, 1H), 5.54 (s, 1H), 5.39-5.29 (m, 2H), 4.75 (d, J=8.9 Hz, 1H), 4.64 (dd, J=14.9, 7.0 Hz, 1H), 4.52-4.45 (m, 2H), 4.30 (dd, J=14.9, 5.0 Hz, 1H), 3.94 (dd, J=11.0, 4.2 Hz, 1H), 3.83-3.78 (m, 1H), 3.28 (qd, J=6.5, 2.4 Hz, 2H), 2.52 (s, 3H), 2.37 (d, J=14.2 Hz, 1H), 2.27-2.12 (m, 6H), 2.00 (q, J=6.7 Hz, 4H), 1.87-1.73 (m, 5H), 1.60 (t, J=7.5 Hz, 3H), 1.28 (q, J=6.8, 4.8 Hz, 27H), 0.94 (s, 9H), 0.90-0.86 (m, 4H). ¹³C NMR (101 MHz, Chloroform-d) δ 173.71, 172.78, 172.58, 172.31, 150.37, 148.57, 137.32, 131.47, 131.29, 130.04, 129.71, 129.65, 128.20, 71.14, 59.87, 58.65, 57.50, 43.52, 38.67, 36.84, 35.01, 34.87, 33.53, 31.91, 29.77, 29.72, 29.53, 29.33, 29.28, 29.15, 27.23, 27.18, 26.38, 25.92, 25.80, 22.69, 16.07, 14.13. HRMS (ESI, m/z): [M+H]⁺ calcd for C₄₄H₇₀N₅O₅S⁺ 780.509218. found 780.51312.

DeFer-3: ¹H NMR (400 MHz, Chloroform-d) δ 8.68 (s, 1H), 7.44-7.30 (m, 5H), 6.22 (d, J=8.8 Hz, 1H), 5.70-5.62 (m, 1H), 5.40-5.28 (m, 2H), 4.73 (t, J=8.1 Hz, 1H), 4.59-4.49 (m, 3H), 4.35 (dd, J=15.0, 5.3 Hz, 1H), 4.09 (d, J=11.4 Hz, 1H), 3.60 (dd, J=11.3, 3.6 Hz, 1H), 3.20 (q, J=6.8 Hz, 2H), 2.52 (s, 4H), 2.26-2.09 (m, 6H), 2.00 (d, J=6.5 Hz, 5H), 1.60 (dt, J=14.0, 7.1 Hz, 5H), 1.46 (p, J=7.3 Hz, 2H), 1.27 (d, J=7.7 Hz, 20H), 0.94 (s, 9H), 0.90-0.85 (m, 3H). ¹³C NMR (101 MHz, Chloroform-d) δ 173.65, 173.33, 171.92, 170.83, 150.30, 148.49, 138.13, 131.59, 130.98, 130.02, 129.73, 129.53, 128.10, 70.03, 58.52, 57.52, 56.84, 43.24, 39.22, 36.84, 36.13, 36.06, 34.94, 31.91, 29.77, 29.73, 29.71, 29.53, 29.33, 29.32, 29.30, 29.17, 27.23, 27.19, 26.42, 26.15, 25.80, 25.07, 22.69, 16.06, 14.13. HRMS (ESI, m/z): [M+Na]⁺ calcd for C₄₆H₇₃N₅O₅SNa⁺ 830.52246.3288. found 830.52122.

### Example 2: Discovery of Ferritin-Targeting Protac DeFer-2

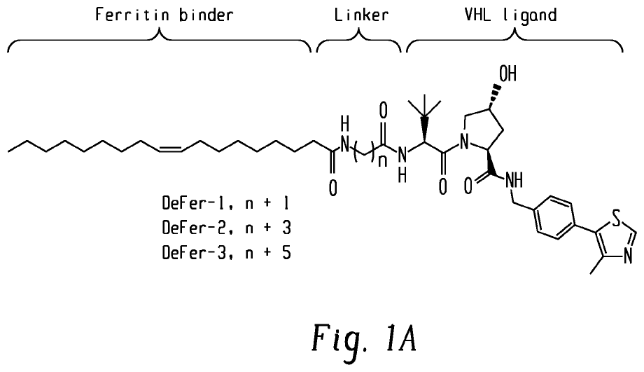

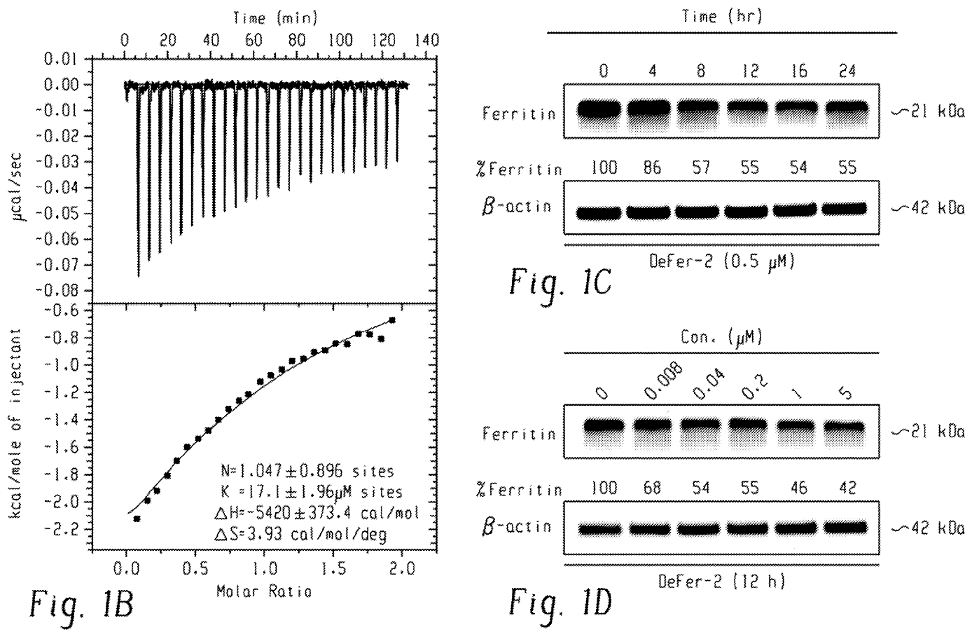

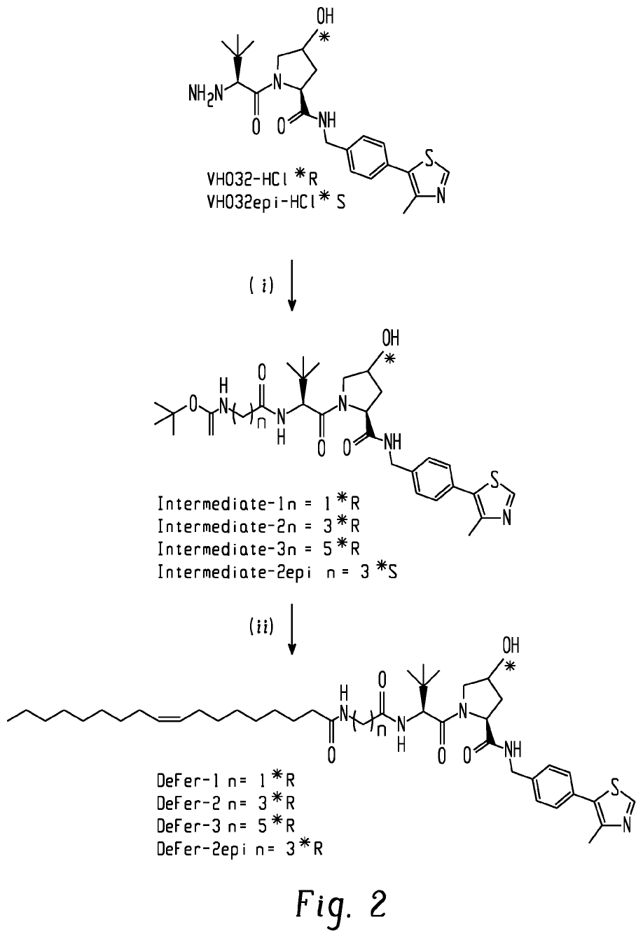

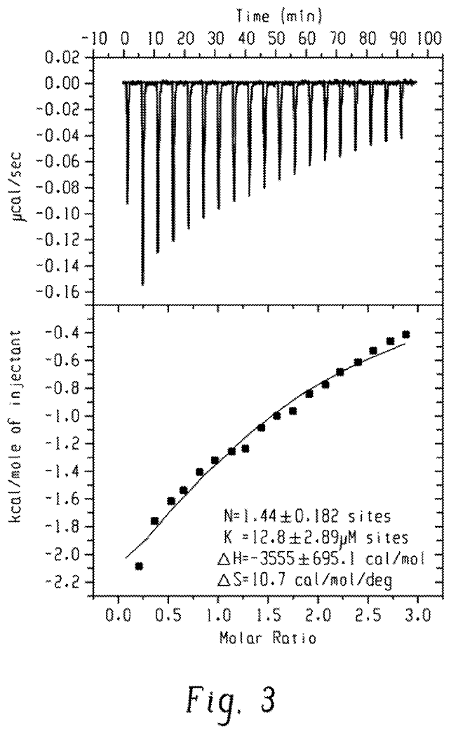

Three OA-based PROTACs, DeFer-1, DeFer-2, and DeFer-3 (FIG. 1A), were generated through two steps of facile amidation reaction (FIG. 2). The obtained chimeras and relevant intermediates were confirmed by ¹H- and ¹³C-nuclear magnetic resonance (NMR) spectra as well as high-resolution mass spectra (HRMS). Prior to the validation of ferritin degradation, the binding affinity of oleic acid and the synthesized chimeras to ferritin was confirmed through isothermal titration calorimetry (ITC) assay. According to the kinetics parameters, OA could bind to ferritin with a K_{d} of 12.8±2.89 μM (FIG. 3). Since the relatively low solubility of the PROTAC molecules in the ITC buffer, inverse titration was performed by leaving other conditions unchanged. Taking DeFer-2 as an example, the binding constant (K_{d}=17.1±1.96 μM) was comparable to the free counterpart (FIG. 1B), indicating that the OA-based PROTACs possessed a reasonable affinity to ferritin and the amidation on the carboxyl group showed negligible effect on the binding kinetics.

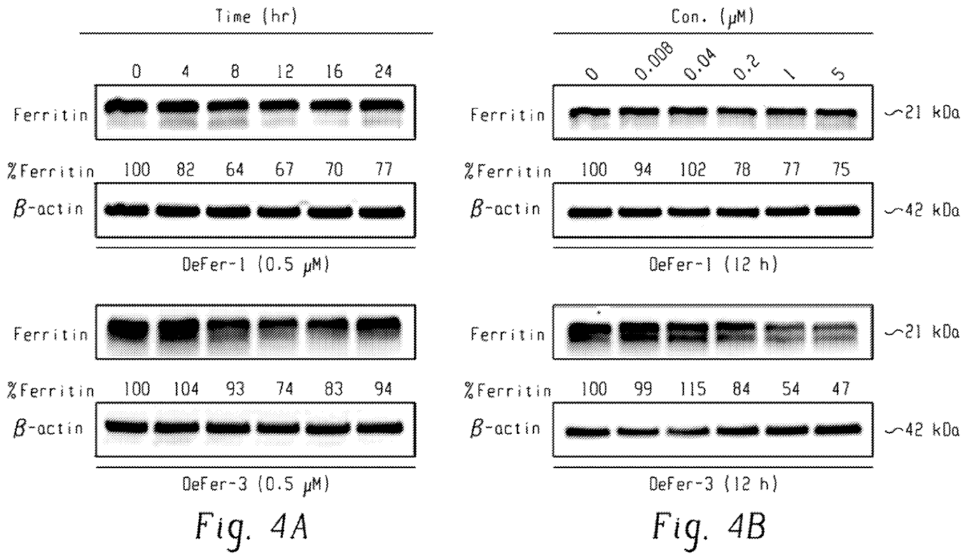

To evaluate the ferritin degradation efficacy, B16F10 melanoma cells were incubated with these PROTACs at a concentration of 0.5 μM for different time points. Even though all three chimeric molecules (DeFer-1, DeFer-2, and DeFer-3) reduced the ferritin expression in a time-dependent manner, DeFer-2 exhibited the most potent degradation capacity with a longer duration of action (FIGS. 1C and 4A). Since all three PROTACs achieved their maximum degradation potency at 12 h, the ferritin level in B16F10 cells treated with various concentrations of PROTACs for 12 h was investigated. As shown in FIGS. 1D and 4B, the ferritin expression in DeFer-2-treated B16F10 cells consistently indicated the most reduction with a maximum of 58% degraded at 5 μM. Collectively, DeFer-2 showed relatively more potent degradation efficacy than both DeFer-1 and DeFer-3. It was reported that the structural characteristics of the linkers between certain ligand pairs play an important role in the final degradation efficiency. To demonstrate the efficacy of ferritin degraders in quickly elevating the intracellular iron stress, as a proof of concept, the DeFer-2 with the best ferritin degradation efficacy was selected for the following studies.

### Example 3: Mechanism of Action of DeFer-2-Mediated Ferritin Degradation

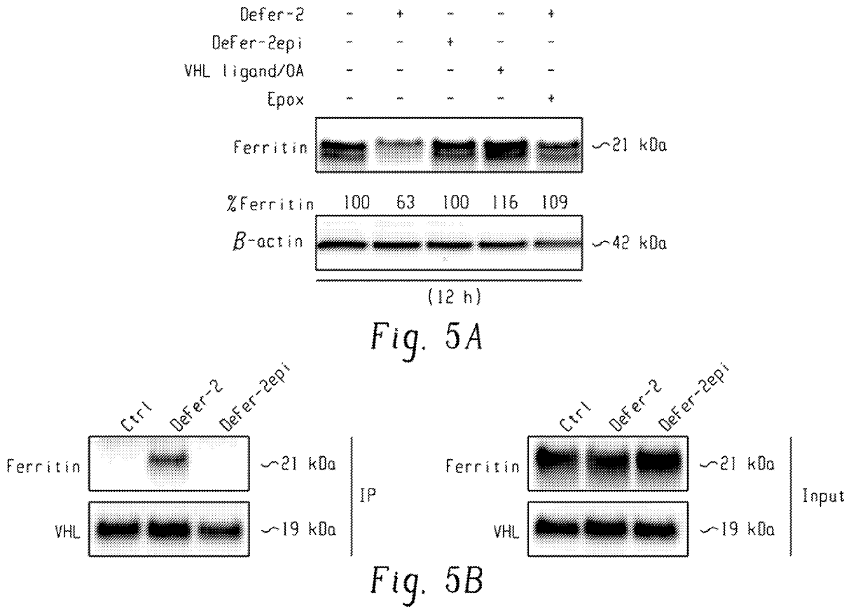

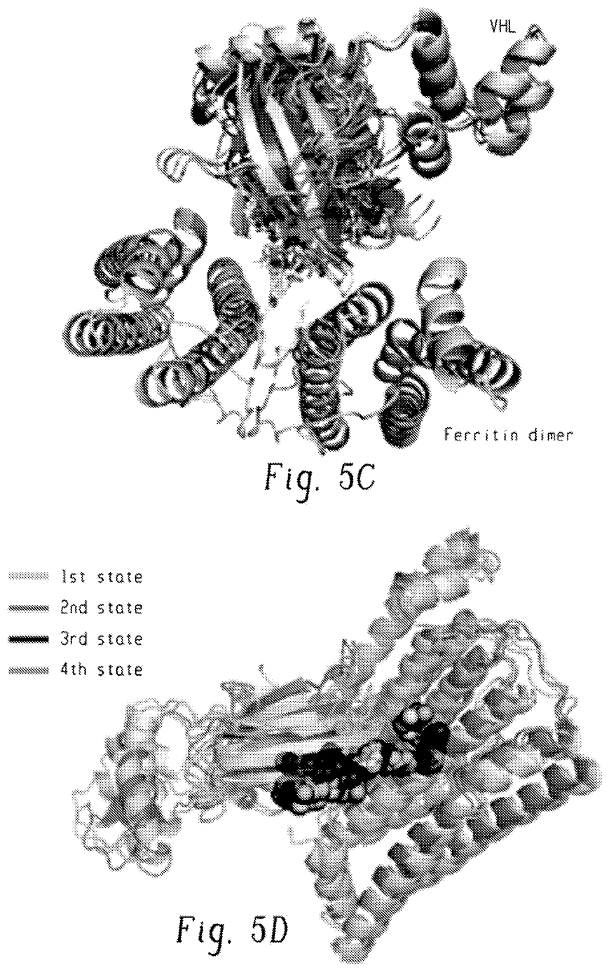

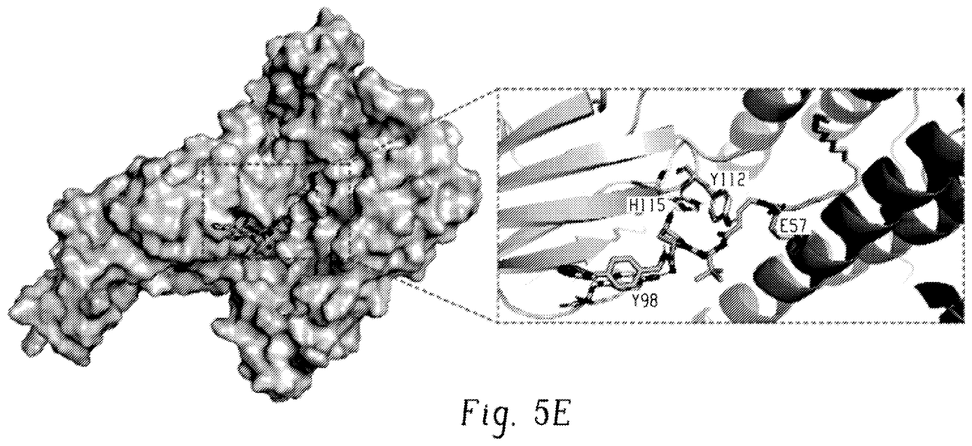

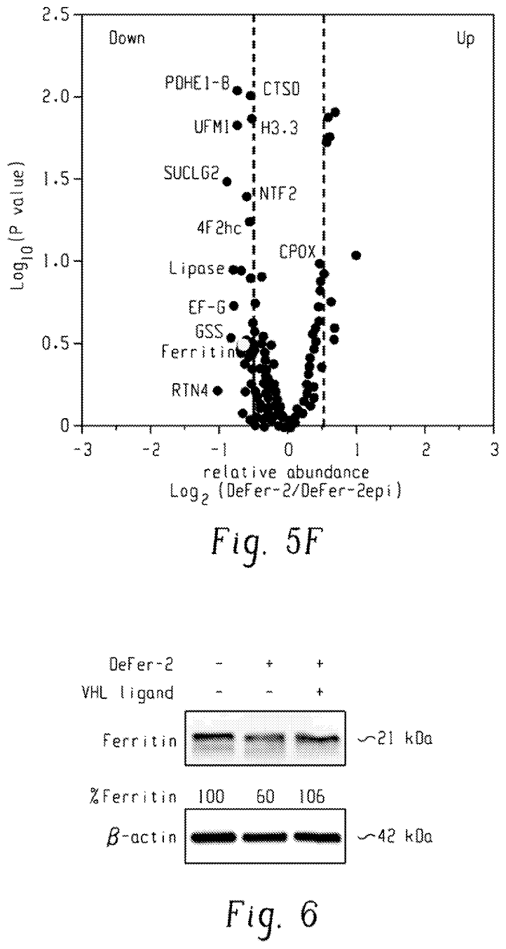

Before evaluating the potential of DeFer-2 to establish iron stress, a range of experiments were conducted to confirm the dependency of DeFer-2-caused ferritin degradation on the UPS. To validate the VHL-recruiting mechanism, an inactive control structure was synthesized that contains an inverted stereocenter in the VHL ligand part, designated DeFer-2epi, according to the same synthesis method of DeFer-2 (FIG. 2). DeFer-2epi shares the identical physicochemical features of DeFer-2 but cannot bind to VHL. As shown in FIG. 5A, DeFer-2 but not DeFer-2epi successfully induced the ferritin degradation, indicating the VHL involvement in DeFer-2-mediated ferritin downregulation. Besides, the decreased ferritin level in the cytoplasm was compromised in the presence of excessive VHL ligands, which also substantiated the VHL-mediated mechanism of action (FIG. 6). Additionally, the combination treatment of VHL ligand and OA at the same concentration failed to reduce the ferritin expression (FIG. 5A). This result demonstrated the importance of DeFer-2 simultaneously binding to VHL and ferritin in this downregulation process. The degradation effect of DeFer-2 was inhibited by the pretreatment of proteasome inhibitor epoxomicin (Epox), suggesting that DeFer-2 induced ferritin degradation in B16F10 cells via the UPS mechanism. Subsequently, it was further verified that DeFer-2 formed the ternary complex with the ferritin and VHL to trigger the degradation process. The co-immunoprecipitation (co-IP) experiment was adopted to determine that DeFer-2 promoted the interaction between ferritin and VHL. The cell lysates were collected from B16F10 cells treated with 5 μM of DeFer-2 or DeFer-2epi for 2 h. After adding the anti-VHL antibody to lysates and the subsequent precipitating procedures, the WB assay was employed to probe the presence of ferritin. The results revealed that DeFer-2 rather than DeFer-2epi could induce ferritin co-precipitated with VHL (FIG. 5B), indicating the ternary complex formation among DeFer-2, ferritin, and VHL.

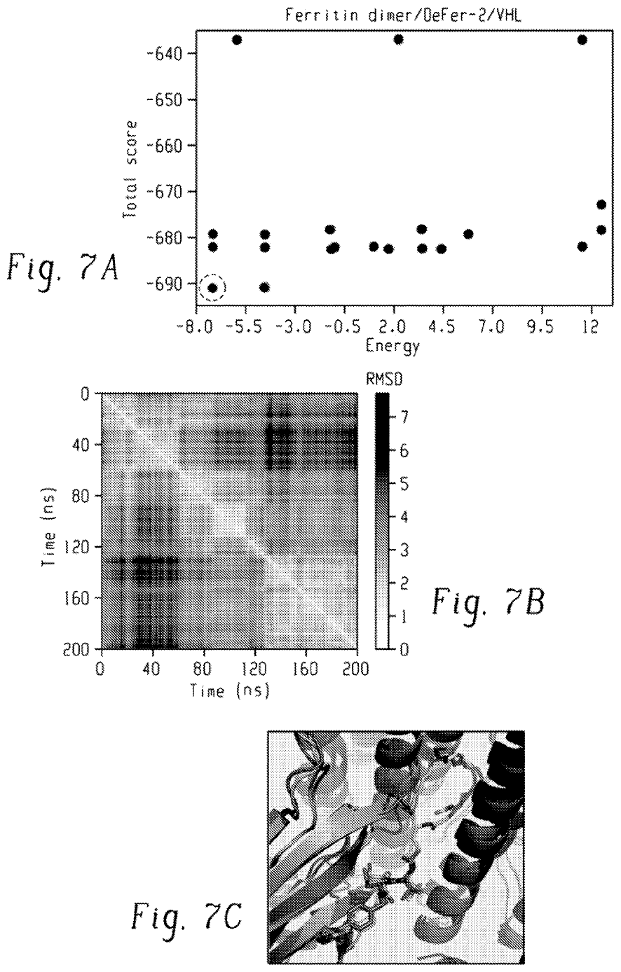

Furthermore, it was tested whether DeFer-2 could form a favorable ternary complex with ferritin dimer and VHL through holistic in silico modeling and simulation. Firstly, based on the Rosetta protein-protein docking framework, various feasible ferritin dimer-VHL interaction modes were established, providing sufficient options for the decoy with binding energy ≤−1.0 and ligand-ligand distance ≤8 Å. Next, the qualified interaction modes were employed to construct the ferritin dimer-DeFer-2-VHL ternary complexes through the conformational alignment of the aminobutyryl linker (FIG. 5C). Among them, the optimal ternary complex structure, which possessed the minimum ligand conformer energy and protein docking score (FIG. 7A), was subjected to molecular dynamics simulation to examine the stability of the relevant conformation. The results showed that the ternary complex structure underwent four stages during the simulation (FIGS. 5D and 7B). In detail, the first steady state lasted about 55 frames (5.5 ns), then the conformation was changed from the second to the third state at approximately 77 frames (7.7 ns). After lasting another 16 frames (1.6 ns), the complex reached a relatively stable status (4th state) at 147 frames, presenting no significant changes until the simulation ended. Moreover, the docking profiles revealed that the oleoyl moiety of DeFer-2 with a U-shaped pattern was well embedded into the cavity at the interface of the ferritin dimer (FIG. 5E). The amide carbonyl group formed a hydrogen bond with residue E57, which serves as the key component of the mineral nucleation site. The VHL ligand moiety occupied the catalytic tunnel of the VHL E3 ligase due to the hydrogen bonds among the hydroxyproline of the ligand, residue Y98, H115, and Y112. Compared to the original crystal structures, the binders of the chimera shared closely resembled interactions and conformations with free counterparts bound to VHL and ferritin dimer (FIG. 7C). Collectively, the modeling data suggested that DeFer-2 held the potential to build a stable ternary complex with VHL and ferritin dimer.

Due to the involvement in several metabolism and signaling pathways, free oleic acid might bind to other enzymes or proteins besides ferritin. To evaluate the selectivity of the ferritin degraders, the quantitative proteomics analysis was conducted to assess the changes in the abundance of whole-cell B16F10 proteins after DeFer-2 treatment. The relative protein abundance changes were compared between DeFer-2- and DeFer-2epi-treated B16F10 cells, and the quantified proteins with an absolute fold-change difference over 1.4 were identified (FIG. 5F). Compared to DeFer-2epi, DeFer-2 could reduce the ferritin levels by 44.1% in B16F10 cells with similar efficiency as indicated by WB results (FIG. 5A). Other proteins with substantially decreased abundance caused by the DeFer-2 treatment included pyruvate dehydrogenase E1 component subunit beta (PDHE1-B, mitochondrial), cathepsin D (CTSD), ubiquitin-fold modifier 1 (UFM1), histone H3.3 (H3.3), succinate-CoA ligase [GDP-forming] subunit beta (SUCLG2, mitochondrial), nuclear transport factor 2 (NTF2), 4F2 cell-surface antigen heavy chain (4F2 hc or SLC3A2), lipase, oxygen-dependent coproporphyrinogen-III oxidase (CPOX, mitochondrial), elongation factor G (EF-G, mitochondrial), glutathione synthetase (GSS), and reticulon (RTN4). Among them, lipase has been reported to hold the OA-binding pocket. However, according to the reported X-ray diffraction analysis (PDB: 1GT6), OA bound within the active site of lipase likely adopts an orientation that the hydrophilic carboxyl group is buried in the interior of the pocket. Coupled with the role of lipase in hydrolyzing fatty acid ester, the OA carboxyl group that serves as the action site could hardly bind to the superficial sites of lipase or protrude from the pocket. In the case of DeFer-2 binding to lipase, it would not be easy to form the ternary complex among the PROTAC, VHL, and lipase, which is needed for ubiquitination. Hence, we speculated that the reduced expression levels of lipase in B16F10 cells after DeFer-2 could be a consequence of ferritin degradation. Except for lipase, the substantially downregulated proteins either associate with the intracellular redox homeostasis, such as SLC3A2 and GSS, or reside in mitochondria, ranging from PDHE1-B and SUCLG2 to CPOX and EF-G. Since the reduced ferritin frequently induces reactive oxygen species (ROS) elevation, the downregulation of SLC3A2 and GSS could be regarded as a consequence of ferritin degradation. Furthermore, many studies reported the positive connections between ROS elevation and mitochondria damage. Therefore, the variation in these mitochondrial proteins could be interpreted as the response of mitochondria to ferritin degradation-mediated oxidative stress. Collectively, it is demonstrated that DeFer-2 has a strong on-target effect to degrade the ferritin to induce the disruption of homeostasis in the redox and mitochondria that are considered as downstream impact of sharp elevation of intracellular iron ions.

### Example 4: Defer-2 Rapidly Elevates Intracellular Free Iron Content to Trigger Pyroptosis in Cancer Cells

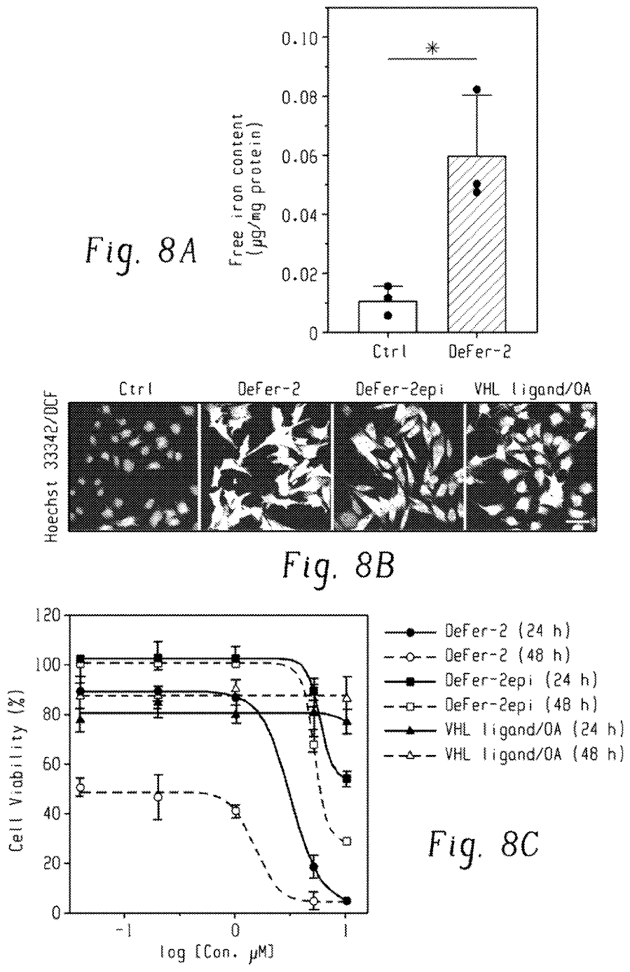

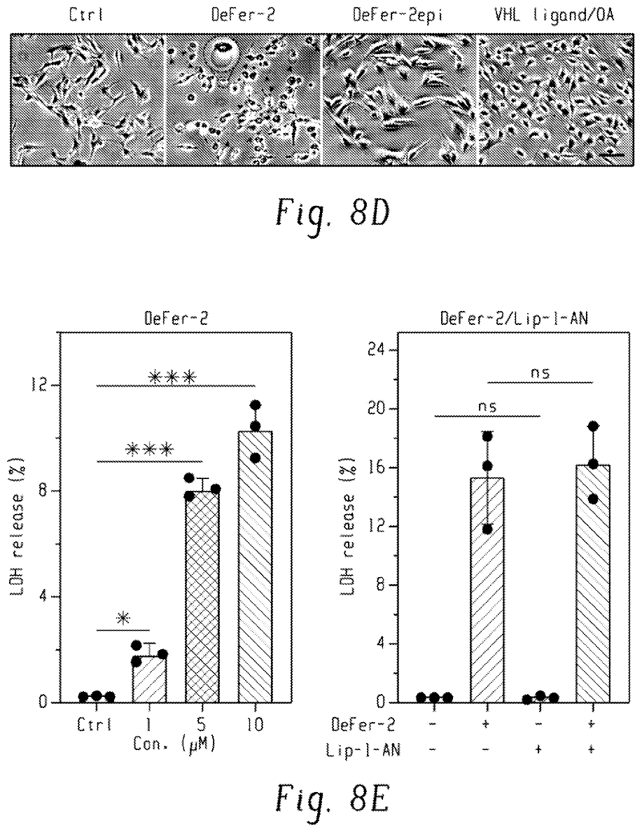

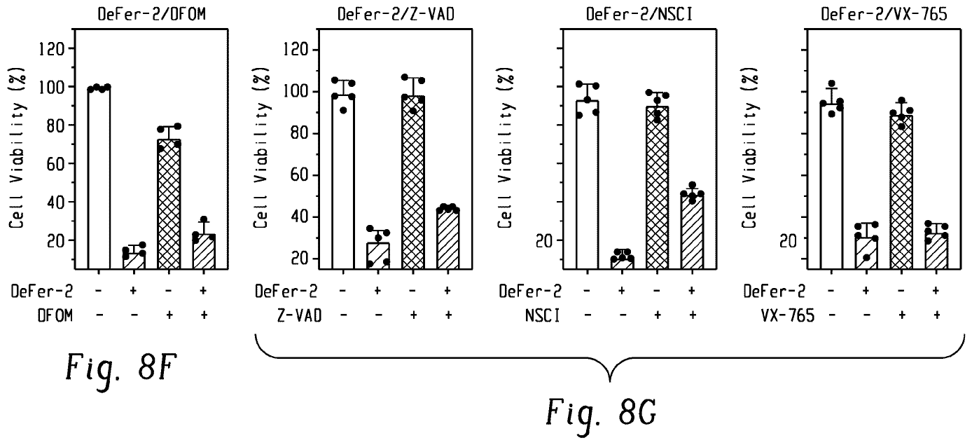

Following the validation of DeFer-2-mediated ferritin degradation via the UPS mechanism, the ability of DeFer-2 to induce an iron overload status inside cancer cells was confirmed. Without considering the valence state of Fe, the intracellular iron content can be generally classified into two categories: protein-bound iron and free iron ions. In this case, the key to detecting the free iron ions is to separate the aqueous phase from the biomacromolecules and organelles in the cytoplasm without affecting the protein-bound iron contents. Based on a previously reported method, we added the ultrapure water to the DeFer-2-treated B16F10 cells to collect all the cytoplasmic inclusions. Then, ultrafiltration was adopted to obtain the free iron ions-containing filtrates. Finally, the detected iron contents were normalized to the total cellular protein of each sample. Due to the favorable ferritin degradation efficacy, the free iron content in B16F10 cells exhibited a significant elevation with the treatment of 5 μM of DeFer-2 for 8 h (FIG. 8A), indicating the accumulation of iron ions in the cytoplasm. Besides, DeFer-2 treatment induced a sharp rise in the ROS level compared to DeFer-2epi and the combination of VHL ligand and OA (FIG. 8B). Since intracellular iron content is closely related to oxidative stress, this ROS change further substantiated the increased free iron ion concentration. Taken together, it was demonstrated that the ferritin-destructive capacity of DeFer-2 created an iron excess stress in malignant cells.

Encouraged by the successful establishment of iron overload, we started to explore the response of cancer cells to this DeFer-2-induced stress state. Firstly, the effect of iron stress on cell viability was investigated using a cell counting kit 8 (CCK-8) assay. Upon treatment at various concentrations, DeFer-2 caused severe cytotoxicity to B16F10 cells with half maximal inhibitory concentration (IC₅₀) values of 3.1 μM and 1.5 μM for 24 h and 48 h, respectively (FIG. 8C). In contrast, the combination of VHL ligand and OA showed little cytotoxicity against B16F10 cells, while DeFer-2epi only exhibited moderate toxicity at higher concentrations. More importantly, dying cancer cells after treatment with DeFer-2 showed obvious swelling and characteristic membrane blebbing, presenting a pyroptosis-like morphology (FIG. 8D). Besides, DeFer-2 triggered the release of lactate dehydrogenase (LDH), a typical indicator for the pyroptosis-induced intracellular content release, in a concentration-dependent manner (FIG. 8E), further confirming the occurrence of the pyroptotic process in B16F10 cells. It has been widely reported that excessive iron ions accumulated in the cytoplasm would frequently initiate ferroptosis, another programmed cell death with increased membrane permeability and cellular contents release. Whereas, the phenomenon presented here is typical pyroptosis rather than ferroptosis. To further substantiate these findings, a ferroptosis inhibitor was co-incubated with DeFer-2 and cancer cells. Upon treatment with the ferroptosis inhibitor-liproxstatin-1 analog (Lip-1-AN) for 4 h, the subsequent LDH caused by DeFer-2 exhibited no evident reduction in B16F10 cells (FIG. 8E). Collectively, the DeFer-2-induced iron overload could initiate the pyroptosis process and induce a programmed cancer cell death process.

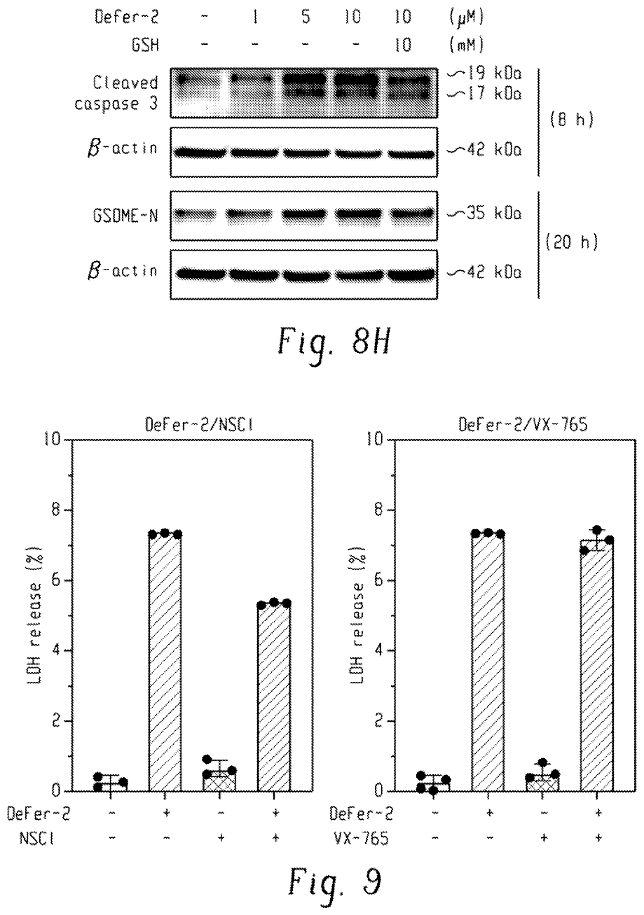

As for further exploration of the underlying mechanism, the role of iron ions in this unexpected phenomenon was verified. After incubating B16F10 cells with DeFer-2 for 4 h, the addition of deferoxamine mesylate (DFOM, an iron-chelating agent) could significantly suppress the cytotoxicity of DeFer-2, indicating the key role of free iron ions in the antiproliferation activity (FIG. 8F). Based on the cell viability profiles, DeFer-2-induced cell death could be compromised by Z-VAD(OMe)-FMK (Z-VAD, a pan-caspase inhibitor) and NSCI (a selective caspase-3 inhibitor), respectively, while that remained unchanged in the presence of caspase-1 inhibitor, VX-765 (FIG. 8G). Similarly, the LDH release from DeFer-2-treated B16F10 cells was efficiently blocked by NSCI but barely affected by VX-765 (FIG. 9). These results indicated a potential mechanism that DeFer-2 initiated the iron ion-caspase 3-mediated pyroptosis in B16F10 cells.

Theoretically, after being cleaved by the upstream enzyme, two subunits, p17 and p12, released from the caspase-3 precursor, undergo dimerization and then shear GSDME, leading to the generation of the active N-terminal domain (GSDME-N). As the final executor of pyroptosis, GSDME-N will be inserted into the plasma membrane to form pores, resulting in swelling, lysis, and intracellular contents release. The activation of caspase 3 in B16F10 cells after DeFer-2 treatment was detected. The western blotting results showed that DeFer-2 increased the level of cleaved caspase 3 in a dose-dependent manner (FIG. 8H). Moreover, DeFer-2-treated B16F10 cells exhibited substantially reduced activated caspase 3 with glutathione (GSH) pretreatment, indicating the key role of ROS in the ferritin degrader-caused caspase 3 activation. Consistently, DeFer-2 could facilitate the cleavage of GSMDE and thus enhance the generation of GSDME-N in B16F10 cells, which was also compromised by GSH pretreatment. Several studies have reported the connections between severe ROS elevation in the cytoplasm and caspase 3 activation. Coupled with the proteomic analysis (FIG. 5F), it was speculated that the increased ROS concentration damaged mitochondria or acutely disrupted its homeostasis, leading to cytochrome c release and subsequent caspase 3 cleavage. In the condition with sufficient GSDME expression, the cancer cells tend to undergo pyroptosis rather than apoptosis. Collectively, these data confirmed that DeFer-2 was able to initiate the caspase 3-GSDME-mediated pyroptosis in cancer cells through free iron ion accumulation and ROS elevation.

### Example 5: ADeFer-2 Induces Cytotoxicity In Vitro and Inhibits Tumor Growth In Vivo

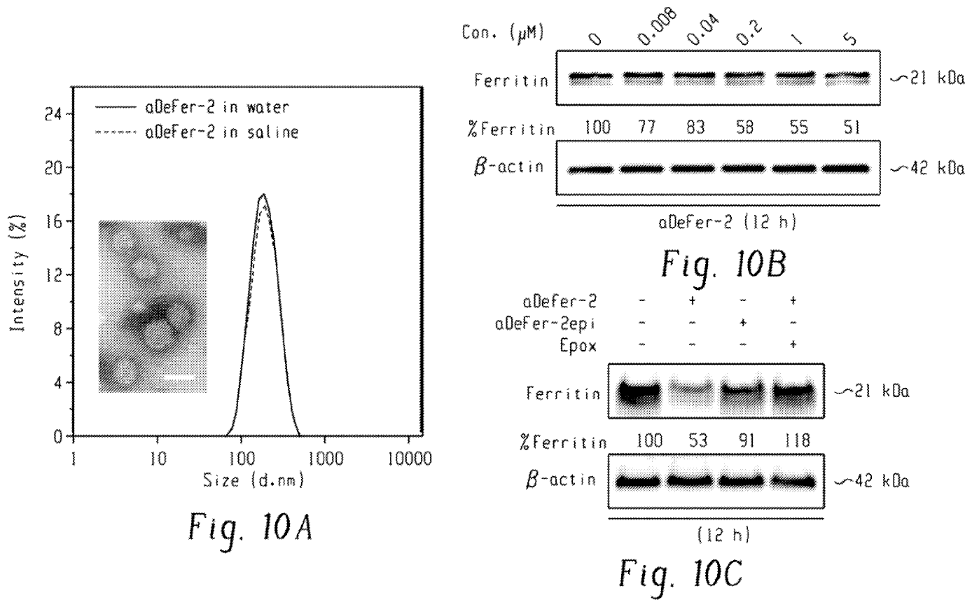

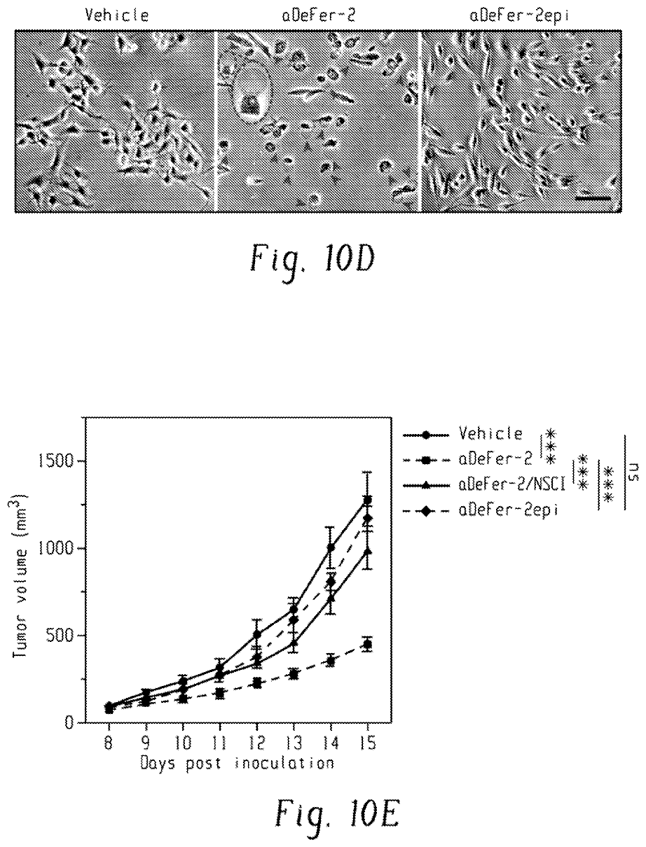

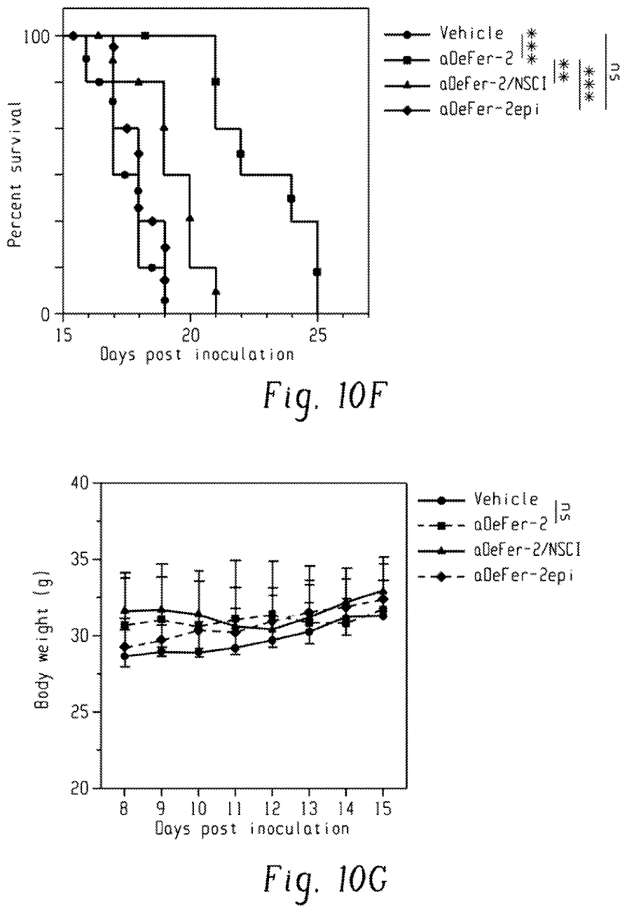

The encouraging in vitro results prompted tapping into the potential of DeFer-2 for in vivo anticancer treatment. However, this chimeric molecule may have a relatively large molecular weight, poor solubility in water, and limited capability to cross physiological barriers, which could significantly compromise its in vivo pharmacokinetics profile and limit the accumulation at the diseased sites or cellular compartments. Therefore, prior to the evaluation of ferritin degrader-mediated treatment efficacy in tumor-bearing mice, a tailored drug formulation for DeFer-2 was prepared based on its structural and physicochemical characteristics. Albumin was used to encapsulate DeFer-2 and provide an albumin-based nano-formulation. Since albumin serves as the natural carrier for fatty acids and other hydrophobic molecules, the oleic acid-based PROTACs possess a natural affinity to albumin, which is beneficial to loading DeFer-2 to the nanoparticles. Further, the rapidly proliferating malignant cells usually have a high demand on taking up and metabolizing albumin to supplement amino acids and energy, which endow the albumin nanoparticle with certain tumor-targeting capacities. Additionally, nano-sized albumin formulations might achieve selective accumulation at the tumor site through the enhanced permeability and retention (EPR) effect, a phenomenon that abnormal vasculature in tumor tissue facilitates the extravasation of macromolecules. Collectively, encapsulating DeFer-2 into albumin nanoparticles holds the potential to selectively transport DeFer-2 to the tumor site. During the formulation optimization, because there are seven potential binding pockets for fatty acid in albumin, the molar ratios between albumin and DeFer-2 were screened, ranging from 1:7 to 1:1, based on the size and stability of the resulting nanoparticles. The favorable ratios were 1:2 and 1:1 within the conditions used herein. Based on the consideration of drug loading efficiency, 1:2 was selected as the optimal ratio between albumin and DeFer-2 to prepare the final nanoparticles. As indicated by dynamic light scattering (DLS), the obtained nanoparticle albumin-DeFer-2, termed aDeFer-2, had a narrow size distribution (polydispersity index (PDI)=0.115±0.014 and 0.144±0.021) with an average size of 158.8±9.8 nm and 154.3±14.4 nm in ultrapure water and saline, respectively (FIG. 10A). Besides, aDeFer-2 displayed a uniform spherical structure under the transmission electron microscope (TEM). The drug loading capacity of aDeFer-2 was 1.86±0.08%, and the encapsulation efficiency was 80.5±3.6%. To investigate the clinical translation capability of aDeFer-2 as an off-the-shelf product, \ the sizes and PDIs of aDeFer-2 in water were determined before lyophilization and resuspended in saline after lyophilization. There were no obvious changes in size and PDI before and after lyophilization (FIG. 11A), indicating the good stability of aDeFer-2 against lyophilization. Furthermore, aDeFer-2 incubated in water could maintain a relatively stable size (PDI<0.3) within 10 days (FIG. 11B). aDeFer-2 was also incubated in PBS containing 10% fetal bovine serum (FBS), a medium to mimic plasma in vitro, to test the release profiles of DeFer-2 from aDeFer-2. As shown in FIG. 11C, the amount of released DeFer-2 from the albumin nanoparticle rapidly increased and reached a maximum of 42.8% at 8 h after incubation. After that, the kinetic curve of the DeFer-2 release approached flat, presenting a similar release behavior as the commercial Abraxane®. As a control, a DeFer-2epi-loaded albumin nanoparticle, termed aDeFer-2epi was prepared, which exhibited comparable size (154.7±10.1 nm in ultrapure water and 161.2±13.7 in saline) and PDI (0.112±0.017 in ultrapure water and 0.136±0.019 in saline) to aDeFer-2 (FIG. 11D).

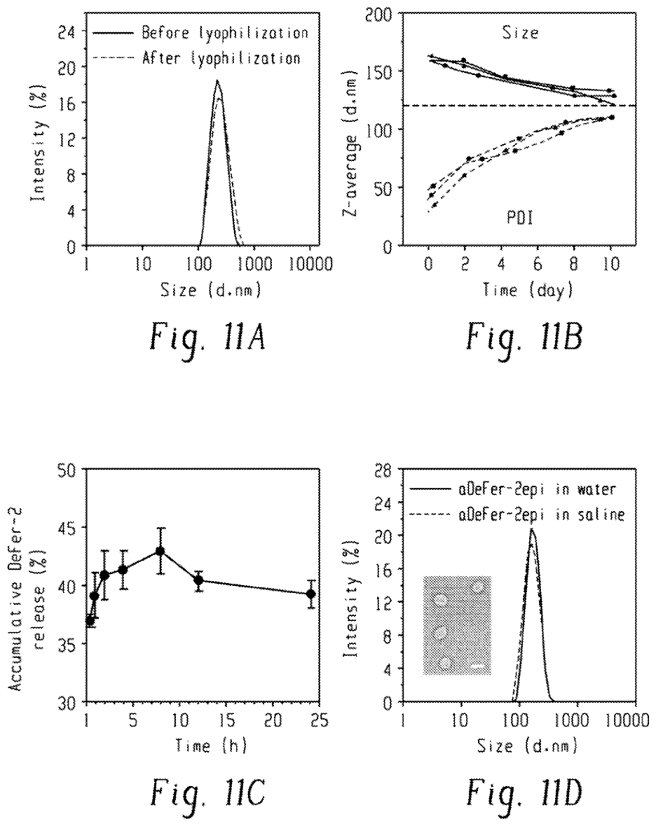

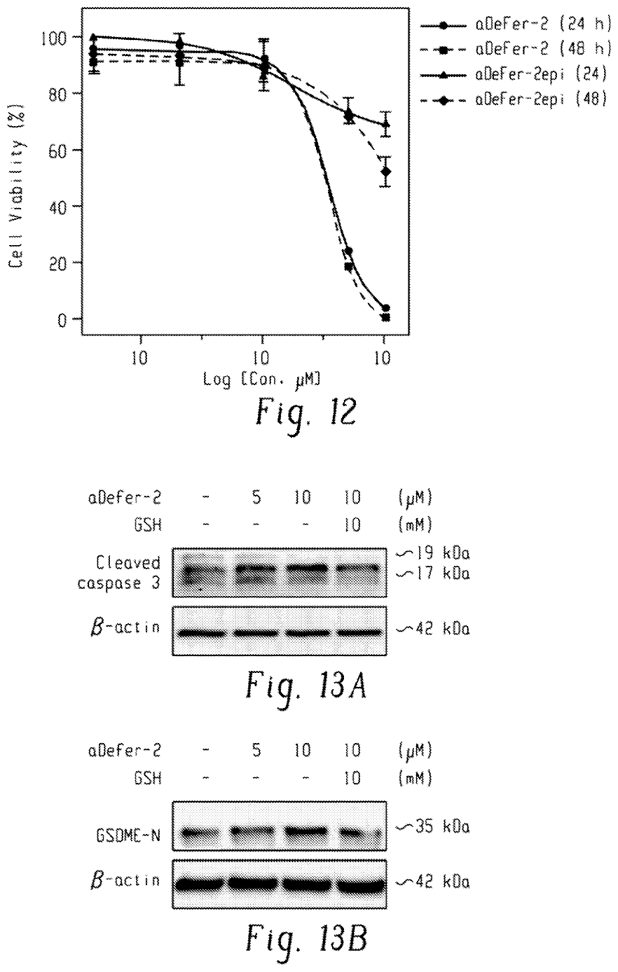

Consistent with DeFer-2, aDeFer-2 maintained excellent ferritin degradation potency in B16F10 cells for 12 h treatment (FIG. 10B). At the same concentration, aDeFer-2epi failed to induce ferritin downregulation (FIG. 10C). More importantly, pretreatment with Epox also compromised the aDeFer-2-mediated ferritin degradation, indicating the same mechanism of action as free DeFer-2. The CCK-8 assays showed that aDeFer-2 but not aDeFer-2epi caused severe cytotoxicity against B16F10 cells (FIG. 12). According to the mechanism validation, aDeFer-2 enhanced the cleavage of caspase 3 in cancer cells through a ROS-mediated course (FIG. 13A), leading to an increased GSDME-N level (FIG. 13B). Coupled with the morphology observation of aDeFer-2-treated B16F10 cells (FIG. 10D), we confirmed that encapsulation of the ferritin degrader into albumin nanoparticles had negligible effects on its bioactivity.

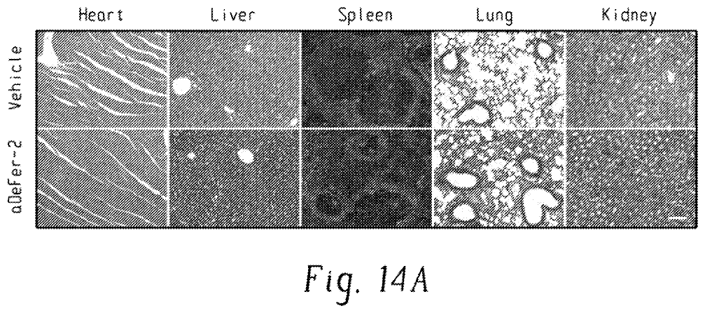

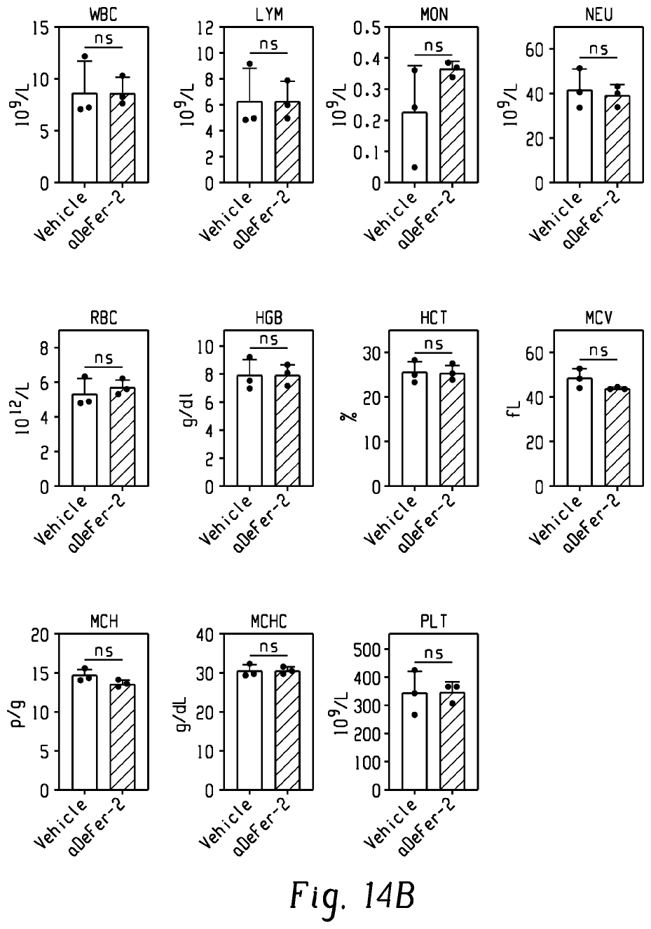

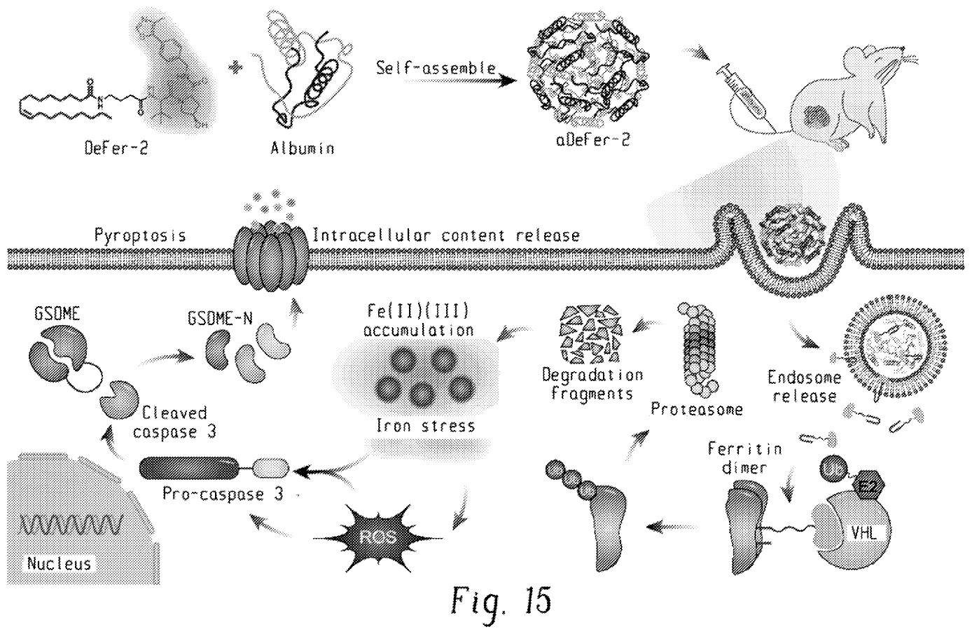

Equipped with this nano-formulation, the therapeutic effect of DeFer-2 was verified in the murine model bearing the subcutaneous B16F10 tumor through intravenous (i.v.) injection. After the tumor volume reached approximately 100 mm³, mice were randomly divided into three groups (n=6), which were administered with the albumin vehicle, aDeFer-2 (10 mg/kg), aDeFer-2epi (10 mg/kg) and aDeFer-2 (10 mg/kg) plus intratumoral injection of caspase 3 inhibitor NSCI (0.4 mg/kg) every other day four times. The treatment efficacy among all groups was determined by comparing the tumor volumes and survival of the tumor-bearing mice. On day 15 post-inoculation, compared to the albumin vehicle group with an average tumor volume of over 1000 mm³, aDeFer-2 showed a substantial inhibitory effect on tumor growth with an average tumor volume of 444 mm³. In contrast, aDeFer-2epi failed to suppress the tumor growth, and the average tumor volume reached 1164 mm³. Consistent with in vitro results, the combination with NSCI could significantly compromise the suppression potency of aDeFer-2, and the average tumor volume in this group reached 982 mm³ on day 15 (FIG. 10E). Notably, the excellent therapeutic potential of aDeFer-2 endowed mice with significantly prolonged survival time (FIG. 10F). The comparatively short median survival extension in the group treated with aDeFer-2 and NSCI might owe to the blockage of aDeFer-2-mediated pyroptosis. The negligible treatment efficacy of aDeFer-2epi suggested that aDeFer-2 realized tumor suppression through a PROTAC mechanism based on in vitro explorations. Moreover, the average body weights of mice treated with aDeFer-2 showed negligible variation in comparison with vehicle-treated mice, indicating the favorable systemic biosafety profile of the aDeFer-2 (FIG. 10G). To further systemically evaluate the biosafety profiles of aDeFer-2, hematoxylin and eosin (H&E) staining was used to determine the histological appearance of the major organs, including the heart, liver, spleen, lung, and kidney. aDeFer-2 administration caused no obvious pathological damage in any of these major organs (FIG. 14A). In addition, we further performed the whole blood cell counts to investigate if there was any change in the number of blood cells that could serve as an indication of severe toxicity. The hematological analysis also demonstrated no significant change in all tested blood cells (FIG. 14B), suggesting that the aDeFer-2 did not induce systemic toxicity.

### DISCUSSION

Described herein is a facile chemico-biological tool to induce intracellular iron overload stress. Given the excellent capacity of ferritin to store the excessive iron ions in the cellular metabolism, it was hypothesized that rapid degradation of ferritin would induce the intracellular accumulation of accessible iron ions. The PROTAC platform was used to design the VHL-recruiting degraders for the seemingly “undruggable” ferritin using the OA that assists the mineralization of iron ions in the cytoplasm as the binder. As a result, the obtained DeFer-2 with a favorable ferritin degradation potency was demonstrated to rapidly increase the intracellular free iron content and elevate the ROS level, leading to potent cytotoxicity against cancer cells. More importantly, the ferritin-targeting PROTAC caused the cancer cells to present the pyroptosis morphologies. With a primary exploration of the underlying mechanism, DeFer-2 triggered the caspase 3-GSDME pathway activation and consequently induced pyroptotic cell death in an iron-dependent manner. Encouraged by the favorable in vitro anticancer performance, a tailored nano-formulation was further developed for the ferritin degrader to unleash the in vivo therapeutic potential based on its structural and physicochemical characteristics. Finally, the resultant albumin-DeFer-2 nanoparticle substantially inhibited tumor growth and prolonged animal survival in the B16F10 tumor models with negligible side effects (FIG. 5).

Described herein is a ferritin-targeting VHL-recruiting PROTAC as a chemico-biological tool for rapidly elevating intracellular free iron content through degrading intracellular ferritin to elevate the intracellular free iron ions. A new programmed cell death mechanism was observed and revealed on how cancer cells responded to intracellular iron excess stress, leading to the development of the PROTAC-based nanomedicine for pyroptosis-mediated anticancer treatment.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A ferritin-targeting PROTAC having the structure
Ferritin Binder-linker-von Hippel-Lindau (VHL) ligand.

2. The PROTAC of claim 1, wherein the Ferritin Binder is a C₁₄ to C₂₄ unsaturated fatty acid, or a haloether anesthetic.

3. The PROTAC of claim 1, wherein the C₁₄ to C₂₄ unsaturated fatty acid is oleate or arachidonate.

4. The PROTAC of claim 1, wherein the haloether anesthetic is halothane, isoflurane, or propofol.

5. The PROTAC of claim 1, wherein the linker has the formula
—NH(CH₂)ₙC(═O)—, wherein n is 1 to 20.

6. The PROTAC of claim 1, wherein the linker has the formula
—(O—CH₂—CH₂)ₘ— or —(O—CH₂—CH₂)ₘ—O—, wherein m is 2 to 20.

7. The PROTAC of claim 1, wherein the VHL ligand is one of the following:

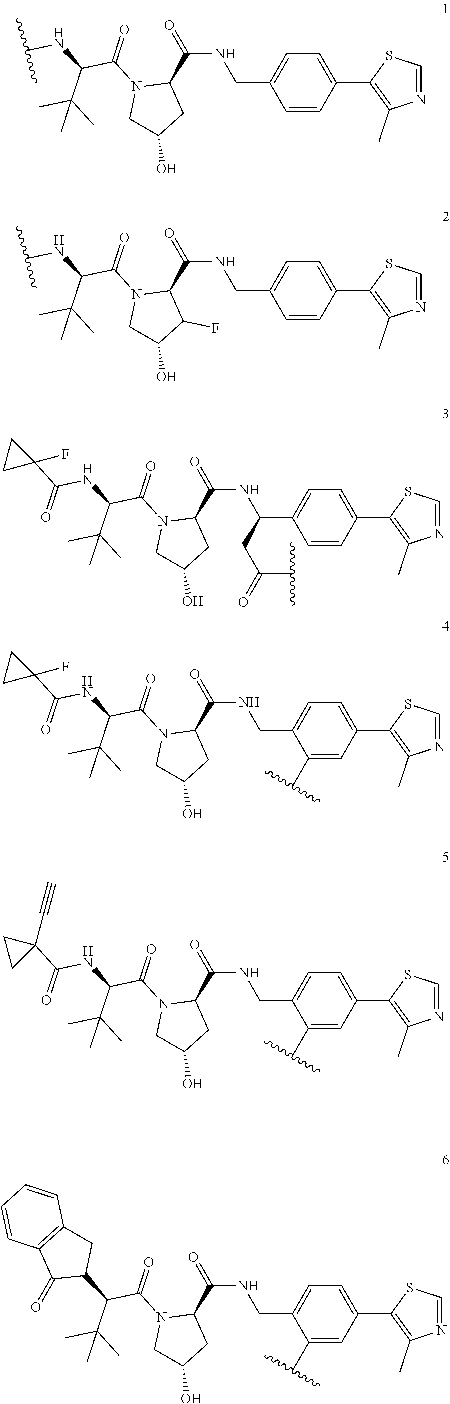 [SMILES: *N[C@@H](C(=O)N1C[C@@H](O)C[C@@H]1C(=O)NCc1ccc(-c2scnc2C)cc1)C(C)(C)C ; *N[C@@H](C(=O)N1C[C@@H](O)C(F)[C@@H]1C(=O)NCc1ccc(-c2scnc2C)cc1)C(C)(C)C ; *C(=O)C[C@@H](NC(=O)[C@H]1C[C@H](O)CN1C(=O)[C@H](NC(=O)C1(F)CC1)C(C)(C)C)c1ccc(-c2scnc2C)cc1 ; *c1cc(-c2scnc2C)ccc1CNC(=O)[C@H]1C[C@H](O)CN1C(=O)[C@H](NC(=O)C1(F)CC1)C(C)(C)C ; *c1cc(-c2scnc2C)ccc1CNC(=O)[C@H]1C[C@H](O)CN1C(=O)[C@H](NC(=O)C1(C#C)CC1)C(C)(C)C ; *c1cc(-c2scnc2C)ccc1CNC(=O)[C@H]1C[C@H](O)CN1C(=O)[C@H](C1Cc2ccccc2C1=O)C(C)(C)C]

8. The PROTAC of claim 1, having the following structure:

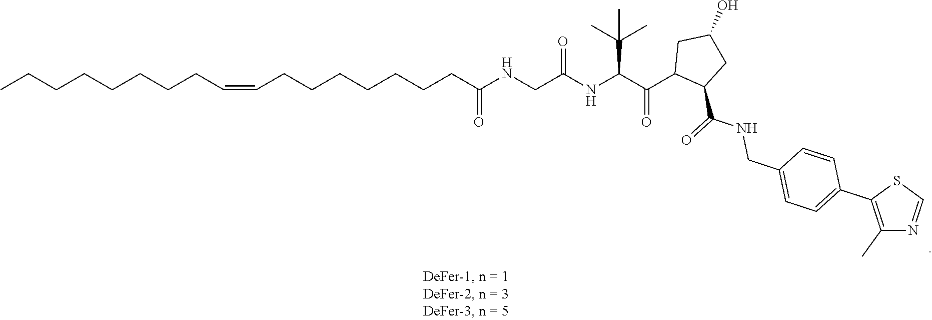 [SMILES: CCCCCCCC/C=C\CCCCCCCC(=O)NCC(=O)N[C@H](C(=O)C1C[C@H](O)C[C@H]1C(=O)NCc1ccc(-c2scnc2C)cc1)C(C)(C)C]

9. A nanoparticle composition comprising the PROTAC of claim 1.

10. The nanoparticle composition of claim 9, comprising albumin.

11. The nanoparticle composition of claim 10, wherein the ratio of the albumin to the ferritin-targeting PROTAC is 1:7 to 1:1, specifically 1:2 to 1:1.

12. The nanoparticle composition of claim 11, wherein the polydispersity index is 0.08 to 0.25, and the average particle diameter is 125 to 200 nm.

13. A pharmaceutical composition comprising the nanoparticle composition of claim 9 and a pharmaceutically acceptable carrier.

14. The pharmaceutical composition of claim 13, in a form for injection.

15. A method of treating a primary or metastatic tumor, a cancer tissue, or a liquid cancer with elevated ferritin expression compared to normal tissues in a subject in need thereof comprises administering the composition of claim 13 to the subject.
